(19) United States
(12) Patent Application Publication
(10) Pub. No.: US 2018/0186807 A1
(43) Pub. Date: Jul. 5, 2018

(54) COMPOUNDS AND USES THEREOF FOR THE MODULATION OF HEMOGLOBIN

(71) Applicant: Global Blood Therapeutics, Inc., South San Francisco, CA (US)

(72) Inventors: Calvin W. Yee, South San Francisco, CA (US); Zhe Li, South San Francisco, CA (US)

(21) Appl. No.: 15/680,794

(22) Filed: Aug. 18, 2017

Related U.S. Application Data
(63) Continuation of application No. 14/542,420, filed on Nov. 14, 2014, now abandoned.
(60) Provisional application No. 61/905,799, filed on Nov. 18, 2013.

(51) Int. Cl. C07D 493/08; C07D 487/08; C07D 401/04; C07C 235/24; C07D 231/40; C07D 213/75; C07D 309/14

(52) U.S. Cl. CPC C07D 493/08; C07D 487/08; C07D 401/04; C07D 309/14; C07D 231/40; C07D 213/75; C07C 235/24

(57) ABSTRACT

Provide herein are compounds and pharmaceutical compositions suitable as modulators of hemoglobin, methods and intermediates for their preparation, and methods for their use in treating disorders mediated by hemoglobin and disorders that would benefit from tissue and/or cellular oxygenation.

### CROSS REFERENCE TO RELATED APPLICATIONS

This application is a continuation application of Ser. No. 14/542,420, filed Nov. 14, 2014, which claims priority to U.S. Provisional Application No. 61/905,799 filed Nov. 18, 2013, the content of which is incorporated herein in its entirety by reference.

### FIELD OF THE INVENTION

This invention provides compounds and pharmaceutical compositions suitable as allosteric modulators of hemoglobin, methods and intermediates for their preparation, and methods for their use in treating disorders mediated by hemoglobin and disorders that would benefit from tissue and/or cellular oxygenation.

### STATE OF THE ART

Sickle cell disease is a disorder of the red blood cells, found particularly among those of African and Mediterranean descent. The basis for sickle cell disease is found in sickle hemoglobin (HbS), which contains a point mutation relative to the prevalent peptide sequence of hemoglobin (Hb).

Hemoglobin (Hb) transports oxygen molecules from the lungs to various tissues and organs throughout the body. Hemoglobin binds and releases oxygen through conformational changes. Sickle hemoglobin (HbS) contains a point mutation where glutamic acid is replaced with valine, allowing HbS to become susceptible to polymerization to give the HbS containing red blood cells their characteristic sickle shape. The sickled cells are also more rigid than normal red blood cells, and their lack of flexibility can lead to blockage of blood vessels. U.S. Pat. No. 7,160,910 discloses compounds that are allosteric modulators of hemoglobin. However, a need exists for additional therapeutics that can treat disorders that are mediated by Hb or by abnormal Hb such as HbS.

### SUMMARY OF THE INVENTION

This invention relates generally to compounds and pharmaceutical compositions suitable as allosteric modulators of hemoglobin. In some aspects, this invention relates to methods for treating disorders mediated by hemoglobin and disorders that would benefit from tissue and/or cellular oxygenation.

In certain aspects of the invention, a compound of formula (I) is provided:

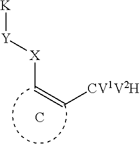

or a tautomer thereof, or a pharmaceutically acceptable salt of each thereof, wherein

### K is:

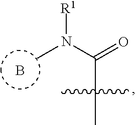

### or K is:

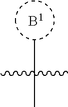

wherein the variables B, B1, and R1 are defined as herein below.

In certain aspects of the invention, a compound of formula (II) is provided:

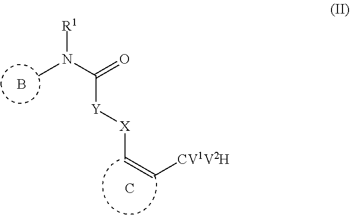

or a tautomer thereof, or a pharmaceutically acceptable salt of each thereof, wherein
ring B is C₆-C₁₀ aryl, C₃-C₈ cycloalkyl, a 5-10 membered heteroaryl containing up to 5 ring heteroatoms or a 4-10 membered heterocycle containing up to 5 ring heteroatoms, wherein the heteroatom is selected from the group consisting of O, N, S, and oxidized forms of N and S, wherein each of the aryl, heteroaryl, cycloalkyl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
each X and Y is independently (CR¹⁰R¹¹)ₑ, O, S, SO, SO₂, or NR¹⁰; e is 1 to 4, preferably 1; each R¹⁰ and R¹¹ independently is hydrogen or C₁-C₃ alkyl optionally substituted with 1-3 halo, OH, or C₁-C₆ alkoxy, or CR¹⁰R¹¹ is C═O, provided that if one of Y and Z is O, S, SO, SO₂, then the other is not CO, and Y and Z are both not heteroatoms or oxidized forms thereof;
ring C is C₆-C₁₀ aryl or a 5-10 membered heteroaryl containing up to 5 ring heteroatoms, wherein the heteroatom is selected from the group consisting of O, N, S, and oxidized forms of N and S, each of which is optionally substituted with 1-4: halo, oxo, —OR², C₁-C₆ alkyl, and/or C₁-C₆ alkoxy, wherein the C₁-C₆ alkyl is optionally substituted with 1-5 halo, C₁-C₆ alkoxy and/or a 4-10 membered heterocycle containing up to 5 ring heteroatoms, wherein the heteroatom is selected from the group consisting of O, N, S, and oxidized forms of N and S;
R¹ is optionally substituted C₁-C₆ alkyl, C₂-C₆ alkenyl, or C₂-C₆ alkynyl, or is C₆-C₁₀ aryl, a 5-10 membered heteroaryl, containing up to 5 ring heteroatoms wherein the heteroatom is selected from the group consisting of O, N, S and oxidized forms of N and S, C₃-C₈ cycloalkyl or a 4-10 membered heterocycle containing up to 5 ring heteroatoms, wherein the heteroatom is selected from the group consisting of O, N, S, and oxidized forms of N and S; and
R² is hydrogen or a prodrug moiety R;
V¹ and V² independently are C₁-C₆ alkoxy; or V¹ and V² together with the carbon atom they are attached to form a ring of formula:

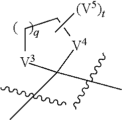

wherein each V³ and V⁴ are independently O, S, or NH, provided that when one of V³ and V⁴ is S, the other is NH, and provided that V³ and V⁴ are both not NH; q is 1 or 2; each V⁵ is independently C₁-C₆ alkyl optionally substituted with 1-3 OH groups, or V⁵ is CO₂R⁶⁰, where each R⁶⁰ independently is C₁-C₆ alkyl or hydrogen; t is 0, 1, 2, or 4; or CV¹V² is C═V, wherein V is O, NOR⁸⁰, or NNR⁸¹R⁸²;
R⁸⁰ is optionally substituted C₁-C₆ alkyl;
R⁸¹ and R⁸² independently are selected from the group consisting of hydrogen, optionally substituted C₁-C₆ alkyl, COR⁸³, or CO₂R⁸⁴;
R⁸³ is hydrogen or optionally substituted C₁-C₆ alkyl; and
R⁸⁴ is optionally substituted C₁-C₆ alkyl.

In certain aspects of the invention, a compound of formula (V) is provided:

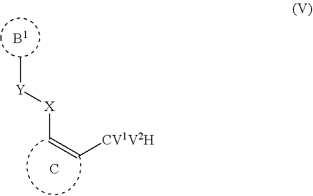

or a tautomer thereof, or a pharmaceutically acceptable salt of each thereof, wherein
ring B¹ is a 5-10 membered heteroaryl containing up to 5 ring heteroatoms or 4-10 membered heterocycle containing up to 5 ring heteroatoms, wherein the heteroatom is selected from the group consisting of O, N, S, and oxidized forms of N and S, wherein at least one of the heteroatoms or oxidized forms thereof is γ to the position where Y is attached to B¹, each of the heteroaryl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
each X and Y is independently (CR¹⁰R¹¹)ₑ, O, S, SO, SO₂, or NR¹⁰; e is 1 to 4, preferably 1; each R¹⁰ and R¹¹ independently is hydrogen or C₁-C₃ alkyl optionally substituted with 1-3 halo, OH, or C₁-C₆ alkoxy, or CR¹⁰R¹¹ is C═O, provided that if one of Y and Z is O, S, SO, SO₂, then the other is not CO, and Y and Z are both not heteroatoms or oxidized forms thereof;
ring C is C₆-C₁₀ aryl or a 5-10 membered heteroaryl containing up to 5 ring heteroatoms, wherein the heteroatom is selected from the group consisting of O, N, S, and oxidized forms of N and S, each of which is optionally substituted with 1-4: halo, oxo, —OR², C₁-C₆ alkyl, and/or C₁-C₆ alkoxy, wherein the C₁-C₆ alkyl is optionally substituted with 1-5 halo, C₁-C₆ alkoxy and/or a 4-10 membered heterocycle containing up to 5 ring heteroatoms, wherein the heteroatom is selected from the group consisting of O, N, S, and oxidized forms of N and S;
R² is hydrogen or a prodrug moiety R;
V¹ and V² independently are C₁-C₆ alkoxy; or V¹ and V² together with the carbon atom they are attached to form a ring of formula:

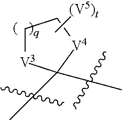

wherein each V³ and V⁴ are independently 0, S, or NH, provided that when one of V³ and V⁴ is S, the other is NH, and provided that V³ and V⁴ are both not NH; q is 1 or 2; each V⁵ is independently C₁-C₆ alkyl optionally substituted with 1-3 OH groups or V⁵ is CO₂R⁶⁰, where each R⁶⁰ independently is C₁-C₆ alkyl or hydrogen; t is 0, 1, 2, or 4; or CV¹V² is C═V, wherein V is O, NOR⁸⁰, or NNR⁸¹R⁸²;
R¹⁰ is optionally substituted C₁-C₆ alkyl;
R⁸¹ and R⁸² independently are selected from the group consisting of hydrogen, optionally substituted C₁-C₆ alkyl, COR⁸³, or CO₂R⁸⁴;
R⁸³ is hydrogen or optionally substituted C₁-C₆ alkyl; and
R⁸⁴ is optionally substituted C₁-C₆ alkyl.

In certain aspects of the invention, a compound of formula (VIII) is provided:

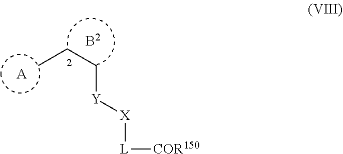

or a tautomer thereof, or a pharmaceutically acceptable salt of each thereof, wherein
ring A is a 5-10 membered heteroaryl, wherein the heteroatom is selected from the group consisting of O, N, S, and oxidized forms of N and S, wherein the heteroaryl is optionally substituted with 1-4: C₁-C₆ alkyl, wherein the C₁-C₆ alkyl is optionally substituted with 1-5 halo, C₁-C₆ alkoxy, and/or C₃-C₁₀ cycloalkyl;
ring B² is a 5-10 membered heteroaryl containing up to 5 ring heteroatoms, wherein the heteroatom is selected from the group consisting of O, N, S, and oxidized forms of N and S, wherein the heteroaryl is optionally substituted with with 1-4: C₁-C₆ alkyl, wherein the C₁-C₆ alkyl is optionally substituted with 1-5 halo, C₁-C₆ alkoxy, and/or C₃-C₁₀ cycloalkyl;
each X and Y is independently CR¹⁰R¹¹, O, S, SO, SO₂, or NR¹⁰; each R¹⁰ and R¹¹ independently is hydrogen or C₁-C₃ alkyl optionally substituted with 1-3 halo, OH, or C₁-C₆ alkoxy, or CR¹⁰R¹¹ is C═O, provided that if one of Y and Z is O, S, SO, SO₂, then the other is not CO, and Y and Z are both not heteroatoms or oxidized forms thereof;
wherein Y is α or β substituted relative to ring B²;
L is joined with X and is a bond or is C₁-C₆ alkylene; and
R¹⁵⁰ is hydrogen, optionally substituted C₁-C₆ alkyl, C₂-C₆ alkynyl, or C₂-C₆ alkynyl, or is C₆-C₁₀ aryl, a 5-10 membered heteroaryl, containing up to 5 ring heteroatoms wherein the heteroatom is selected from the group consisting of O, N, S, C₃-C₈ cycloalkyl or a 4-10 membered heterocycle containing up to 5 ring heteroatoms, wherein the heteroatom is selected from the group consisting of O, N, S.

It is contemplated that a compound of formula (VIII) is useful as an intermediate for making compounds that can modulate hemoglobin.

In further aspects of the invention, a composition is provided comprising any of the compounds described herein, and at least a pharmaceutically acceptable excipient.

In still further aspects of the invention, a method is provided for increasing oxygen affinity of hemoglobin S in a subject, the method comprising administering to a subject in need thereof a therapeutically effective amount of any of the compounds or compositions described herein.

In further aspects of the invention, a method is provided for treating oxygen deficiency associated with sickle cell anemia, the method comprising administering to a subject in need thereof a therapeutically effective amount of any of the compounds or compositions described herein.

### DETAILED DESCRIPTION OF THE INVENTION

### Definitions

It must be noted that as used herein and in the appended claims, the singular forms “a”, “an”, and “the” include plural referents unless the context clearly dictates otherwise. Thus, for example, reference to “a solvent” includes a plurality of such solvents.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition or process consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques. The term “about” when used before a numerical designation, e.g., temperature, time, amount, and concentration, including range, indicates approximations which may vary by (+) or (−) 10%, 5% or 1%.

As used herein, Cₘ-Cₙ, such as C₁-C₁₂, C₁-C₈, or C₁-C₆ when used before a group refers to that group containing m to n carbon atoms.

The term “alkoxy” refers to —O-alkyl.

The term “alkyl” refers to monovalent saturated aliphatic hydrocarbyl groups having from 1 to 12 carbon atoms (i.e., C₁-C₁₂ alkyl) or 1 to 8 carbon atoms (i.e., C₁-C₈ alkyl), or 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “alkenyl” refers to monovalent aliphatic hydrocarbyl groups having from 2 to 12 carbon atoms (i.e., C₂-C₁₂ alkenyl) or 2 to 8 carbon atoms (i.e., C₂-C₈ alkenyl), or 1 to 4 carbon atoms and at least 1 carbon-carbon double bond. This term includes, by way of example, linear and branched hydrocarbyl groups such as vinyl, 2-propenyl, 2-butenyl, and the likes.

The term “alkynyl” refers to monovalent aliphatic hydrocarbyl groups having from 2 to 12 carbon atoms (i.e., C₂-C₁₂ alkynyl) or 2 to 8 carbon atoms (i.e., C₂-C₈ alkynyl), or 1 to 4 carbon atoms and at least 1 carbon-carbon triple bond. This term includes, by way of example, linear and branched hydrocarbyl groups such as ethynyl, propynyl, dimethylpropargyl, 2-butynyl, and the likes.

The term “alkylene” refers to saturated divalent aliphatic hydrocarbyl groups having from 1 to 12 carbon atoms (i.e., C₁-C₁₂ alkylene) or 1 to 8 carbon atoms (i.e., C₁-C₈ alkylene), or 1 to 4 carbon atoms. This term includes, by way of example, linear and branched hydrocarbyl groups such as methylene, ethylene, propylene, 2-methylethylene, and the likes.

The term “aryl” refers to a monovalent, aromatic mono- or bicyclic ring having 6-10 ring carbon atoms. Examples of aryl include phenyl and naphthyl. The condensed ring may or may not be aromatic provided that the point of attachment is at an aromatic carbon atom. For example, and without limitation, the following is an aryl group:

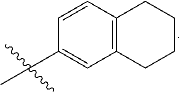 [SMILES: *c1ccc2c(c1)CCCC2]

The term “—CO₂H ester” refers to an ester formed between the —CO₂H group and an alcohol, preferably an aliphatic alcohol. A preferred example included —CO₂R^{E}, wherein R^{E} is alkyl or aryl group optionally substituted with an amino group.

The term “chiral moiety” refers to a moiety that is chiral. Such a moiety can possess one or more asymmetric centers. Preferably, the chiral moiety is enantiomerically enriched, and more preferably a single enantiomer. Non limiting examples of chiral moieties include chiral carboxylic acids, chiral amines, chiral amino acids, such as the naturally occurring amino acids, chiral alcohols including chiral steroids, and the likes.

The term “cycloalkyl” refers to a monovalent, preferably saturated, hydrocarbyl mono-, bi-, or tricyclic ring having 3-12 ring carbon atoms. While cycloalkyl, refers preferably to saturated hydrocarbyl rings, as used herein, it also includes rings containing 1-2 carbon-carbon double bonds. Nonlimiting examples of cycloalkyl include cyclopropyl, cyclobutyl, cyclopentyl, cyclohexyl, cycloheptyl, adamentyl, and the like. The condensed rings may or may not be non-aromatic hydrocarbyl rings provided that the point of attachment is at a cycloalkyl carbon atom. For example, and without limitation, the following is a cycloalkyl group:

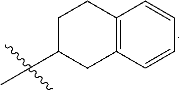 [SMILES: *C1CCc2ccccc2C1]

The term “halo” refers to F, Cl, Br, and/or I.

The term “heteroaryl” refers to a monovalent, aromatic mono-, bi-, or tricyclic ring having 2-16 ring carbon atoms and 1-8 ring heteroatoms selected preferably from N, O, S, and P and oxidized forms of N, S, and P, provided that the ring contains at least 5 ring atoms. Nonlimiting examples of heteroaryl include furan, imidazole, oxadiazole, oxazole, pyridine, quinoline, and the like. The condensed rings may or may not be a heteroatom containing aromatic ring provided that the point of attachment is a heteroaryl atom. For example, and without limitation, the following is a heteroaryl group:

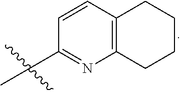 [SMILES: *c1ccc2c(n1)CCCC2]

The term “heterocyclyl” or heterocycle refers to a non-aromatic, mono-, bi-, or tricyclic ring containing 2-12 ring carbon atoms and 1-8 ring heteroatoms selected preferably from N, O, S, and P and oxidized forms of N, S, and P, provided that the ring contains at least 3 ring atoms. While heterocyclyl preferably refers to saturated ring systems, it also includes ring systems containing 1-3 double bonds, provided that the ring is non-aromatic. Nonlimiting examples of heterocyclyl include, azalactones, oxazoline, piperidinyl, piperazinyl, pyrrolidinyl, tetrahydrofuranyl, and tetrahydropyranyl. The condensed rings may or may not contain a non-aromatic heteroatom containing ring provided that the point of attachment is a heterocyclyl group. For example, and without limitation, the following is a heterocyclyl group:

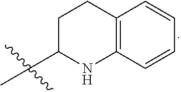 [SMILES: *C1CCc2ccccc2N1]

The term “hydrolyzing” refers to breaking an R^{H}—O—CO—, R^{H}—O—CS—, or an R^{H}—O—SO₂— moiety to an R^{H}—OH, preferably by adding water across the broken bond. A hydrolyzing is performed using various methods well known to the skilled artisan, non limiting examples of which include acidic and basic hydrolysis.

The term “oxo” refers to a C═O group, and to a substitution of 2 geminal hydrogen atoms with a C═O group.

The term “optionally substituted,” unless defined otherwise, refers to a substituted or unsubstituted group. The group may be substituted with one or more substituents, such as e.g., 1, 2, 3, 4 or 5 substituents. Preferably, the substituents are selected from the group consisting of oxo, halo, —CN, NO₂, —N₂+, —CO₂R¹⁰⁰, —SR¹⁰⁰, —SOR¹⁰⁰, —SO₂R¹⁰⁰, —NR¹⁰¹R¹⁰², —CONR¹⁰¹R¹⁰², —SO₂NR¹⁰¹R¹⁰², C₁-C₆ alkyl, C₁-C₆ alkoxy, —CR¹⁰⁰═C(R¹⁰⁰)₂, —CCR¹⁰⁰, C₃-C₁₀ cycloalkyl, C₃-C₁₀ heterocyclyl, C₆-C₁₂ aryl and C₂-C₁₂ heteroaryl, wherein each R¹⁰⁰ independently is hydrogen or C₁-C₈ alkyl; C₃-C₁₂ cycloalkyl; C₃-C₁₀ heterocyclyl; C₆-C₁₂ aryl; or C₂-C₁₂ heteroaryl; wherein each alkyl, cycloalkyl, heterocyclyl, aryl, or heteroaryl is optionally substituted with 1-3 halo, 1-3 C₁-C₆ alkyl, 1-3 C₁-C₆ haloalkyl or 1-3 C₁-C₆ alkoxy groups. Preferably, the substituents are selected from the group consisting of chloro, fluoro, —OCH₃, methyl, ethyl, iso-propyl, cyclopropyl, vinyl, ethynyl, —CO₂H, —CO₂CH₃, —OCF₃, —CF₃ and —OCHF₂.

R¹⁰¹ and R¹⁰² independently is hydrogen; C₁-C₈ alkyl, optionally substituted with —CO₂H or an ester thereof, C₁-C₆ alkoxy, oxo, —CR¹⁰³═C(R¹⁰³)₂, —CCR, C₃-C₁₀ cycloalkyl, C₃-C₁₀ heterocyclyl, C₆-C₁₂ aryl, or C₂-C₁₂ heteroaryl, wherein each R¹⁰³ independently is hydrogen or C₁-C₈ alkyl; C₃-C₁₂ cycloalkyl; C₃-C₁₀ heterocyclyl; C₆-C₁₂ aryl; or C₂-C₁₂ heteroaryl; wherein each cycloalkyl, heterocyclyl, aryl, or heteroaryl is optionally substituted with 1-3 alkyl groups or 1-3 halo groups, or R¹⁰¹ and R¹⁰² together with the nitrogen atom they are attached to form a 5-7 membered heterocycle.

The term “pharmaceutically acceptable” refers to safe and non-toxic for in vivo, preferably, human administration.

The term “pharmaceutically acceptable salt” refers to a salt that is pharmaceutically acceptable.

The term “salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary, and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as caroboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisalfate, mono-, di-, and tribasic phosphate, mesylate, tosylate, and the likes.

The terms “treat”, “treating” or “treatment”, as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting or suppressing the development of the disease or condition, relieving the disease or condition, causing regression of the disease or condition, relieving a condition caused by the disease or condition, or suppressing the symptoms of the disease or condition, and are intended to include prophylaxis. The terms also include relieving the disease or conditions, e.g., causing the regression of clinical symptoms. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “preventing” or “prevention” refer to a reduction in risk of acquiring a disease or disorder (i.e., causing at least one of the clinical symptoms of the disease not to develop in a subject that may be exposed to or predisposed to the disease but does not yet experience or display symptoms of the disease). The terms further include causing the clinical symptoms not to develop, for example in a subject at risk of suffering from such a disease or disorder, thereby substantially averting onset of the disease or disorder.

The term “effective amount” refers to an amount that is effective for the treatment of a condition or disorder by an intranasal administration of a compound or composition described herein. In some embodiments, an effective amount of any of the compositions or dosage forms described herein is the amount used to treat a disorder mediated by hemoglobin or a disorder that would benefit from tissue and/or cellular oxygenation of any of the compositions or dosage forms described herein to a subject in need thereof.

The term “carrier” as used herein, refers to relatively nontoxic chemical compounds or agents that facilitate the incorporation of a compound into cells, e.g., red blood cells, or tissues.

As used herein, a “prodrug” is a compound that, after administration, is metabolized or otherwise converted to an active or more active form with respect to at least one property. To produce a prodrug, a pharmaceutically active compound can be modified chemically to render it less active or inactive, but the chemical modification is such that an active form of the compound is generated by metabolic or other biological processes. A prodrug may have, relative to the drug, altered metabolic stability or transport characteristics, fewer side effects or lower toxicity. For example, see the reference Nogrady, 1985, Medicinal Chemistry A Biochemical Approach, Oxford University Press, New York, pages 388-392. Prodrugs can also be prepared using compounds that are not drugs.

### Compounds

In certain aspects of the invention, a compound of formula (I) is provided:

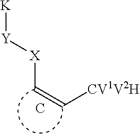

or a tautomer thereof, or a pharmaceutically acceptable salt of each thereof, wherein
K is:

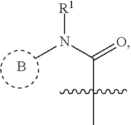

or K is:

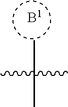

wherein
ring B is C₆-C₁₀ aryl, C₃-C₈ cycloalkyl, a 5-10 membered heteroaryl containing up to 5 ring heteroatoms or a 4-10 membered heterocycle containing up to 5 ring heteroatoms, wherein the heteroatom is selected from the group consisting of O, N, S, and oxidized forms of N and S, wherein each of the aryl, heteroaryl, cycloalkyl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
ring B¹ is a 5-10 membered heteroaryl containing up to 5 ring heteroatoms or 4-10 membered heterocycle containing up to 5 ring heteroatoms, wherein the heteroatom is selected from the group consisting of O, N, S, and oxidized forms of N and S, wherein at least one of the heteroatoms or oxidized forms thereof is γ to the position where Y is attached to B¹, each of the heteroaryl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
each X and Y is independently CR¹⁰R¹¹, O, S, SO, SO₂, or NR¹⁰; each R¹⁰ and R¹¹ independently is hydrogen or C₁-C₃ alkyl optionally substituted with 1-3 halo, OH, or C₁-C₆ alkoxy, or CR¹⁰R¹¹ is C═O, provided that if one of Y and Z is O, S, SO, SO₂, then the other is not CO, and Y and Z are both not heteroatoms or oxidized forms thereof;
ring C is C₆-C₁₀ aryl or a 5-10 membered heteroaryl containing up to 5 ring heteroatoms, wherein the heteroatom is selected from the group consisting of O, N, S, and oxidized forms of N and S, each of which is optionally substituted with 1-4: halo, oxo, —OR², C₁-C₆ alkyl, and/or C₁-C₆ alkoxy, wherein the C₁-C₆ alkyl is optionally substituted with 1-5 halo, C₁-C₆ alkoxy and/or a 4-10 membered heterocycle containing up to 5 ring heteroatoms, wherein the heteroatom is selected from the group consisting of O, N, S, and oxidized forms of N and S;
R¹ is optionally substituted C₁-C₆ alkyl, C₂-C₆ alkenyl, or C₂-C₆ alkynyl, or is C₆-C₁₀ aryl, a 5-10 membered heteroaryl, containing up to 5 ring heteroatoms wherein the heteroatom is selected from the group consisting of O, N, S and oxidized forms of N and S, C₃-C₈ cycloalkyl or a 4-10 membered heterocycle containing up to 5 ring heteroatoms, wherein the heteroatom is selected from the group consisting of O, N, S, and oxidized forms of N and S; and
R² is hydrogen or a prodrug moiety R;
V¹ and V² independently are C₁-C₆ alkoxy; or V¹ and V² together with the carbon atom they are attached to form a ring of formula:

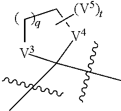

wherein each V³ and V⁴ are independently O, S, or NH, provided that when one of V³ and V⁴ is S, the other is NH, and provided that V³ and V⁴ are both not NH; q is 1 or 2; each V⁵ is independently C₁-C₆ alkyl optionally substituted with 1-3 OH groups, or V⁵ is CO₂R⁶⁰, where each R⁶⁰ independently is C₁-C₆ alkyl or hydrogen; t is 0, 1, 2, or 4; or CV¹V² is C═V, wherein V is O, NOR⁸⁰, or NNR⁸¹R⁸²;
R⁸⁰ is optionally substituted C₁-C₆ alkyl;
R⁸¹ and R⁸² independently are selected from the group consisting of hydrogen, optionally substituted C₁-C₆ alkyl, COR⁸³, or CO₂R⁸⁴;
R⁸³ is hydrogen or optionally substituted C₁-C₆ alkyl; and
R⁸⁴ is optionally substituted C₁-C₆ alkyl.

In certain aspects of the invention, a compound of formula (II) is provided:

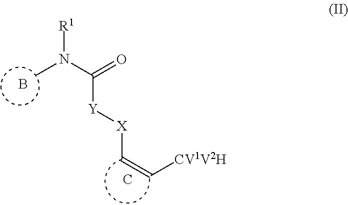

or a tautomer thereof, or a pharmaceutically acceptable salt of each thereof, wherein
ring B is C₆-C₁₀ aryl, C₃-C₈ cycloalkyl, a 5-10 membered heteroaryl containing up to 5 ring heteroatoms or a 4-10 membered heterocycle containing up to 5 ring heteroatoms, wherein the heteroatom is selected from the group consisting of O, N, S, and oxidized forms of N and S, wherein each of the aryl, heteroaryl, cycloalkyl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
each X and Y is independently CR¹⁰R¹¹, O, S, SO, SO₂, or NR¹⁰; each R¹⁰ and R¹¹ independently is hydrogen or C₁-C₃ alkyl optionally substituted with 1-3 halo, OH, or C₁-C₆ alkoxy, or CR¹⁰R¹¹ is C═O, provided that if one of Y and Z is O, S, SO, SO₂, then the other is not CO, and Y and Z are both not heteroatoms or oxidized forms thereof;
ring C is C₆-C₁₀ aryl or a 5-10 membered heteroaryl containing up to 5 ring heteroatoms, wherein the heteroatom is selected from the group consisting of O, N, S, and oxidized forms of N and S, each of which is optionally substituted with 1-4: halo, oxo, —OR², C₁-C₆ alkyl, and/or C₁-C₆ alkoxy, wherein the C₁-C₆ alkyl is optionally substituted with 1-5 halo, C₁-C₆ alkoxy and/or a 4-10 membered heterocycle containing up to 5 ring heteroatoms, wherein the heteroatom is selected from the group consisting of O, N, S, and oxidized forms of N and S;
R¹ is optionally substituted C₁-C₆ alkyl, C₂-C₆ alkenyl, C₂-C₆ alkynyl, C₆-C₁₀ aryl, a 5-10 membered heteroaryl, containing up to 5 ring heteroatoms wherein the heteroatom is selected from the group consisting of O, N, S and oxidized forms of N and S, C₃-C₈ cycloalkyl or a 4-10 membered heterocycle containing up to 5 ring heteroatoms, wherein the heteroatom is selected from the group consisting of O, N, S, and oxidized forms of N and S; and
R² is hydrogen or a prodrug moiety R;
V¹ and V² independently are C₁-C₆ alkoxy; or V¹ and V² together with the carbon atom they are attached to form a ring of formula:

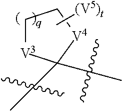

wherein each V³ and V⁴ are independently O, S, or NH, provided that when one of V³ and V⁴ is S, the other is NH, and provided that V³ and V⁴ are both not NH; q is 1 or 2; each V⁵ is independently C₁-C₆ alkyl optionally substituted with 1-3 OH groups, or V⁵ is CO₂R⁶⁰, where each R⁶⁰ independently is C₁-C₆ alkyl or hydrogen; t is 0, 1, 2, or 4; or CV¹V² is C═V, wherein V is O, NOR⁸⁰, or NNR⁸¹R⁸²;
R⁸⁰ is optionally substituted C₁-C₆ alkyl;
R⁸¹ and R⁸² independently are selected from the group consisting of hydrogen, optionally substituted C₁-C₆ alkyl, COR⁸³, or CO₂R⁸⁴;
R⁸³ is hydrogen or optionally substituted C₁-C₆ alkyl; and
R⁸⁴ is optionally substituted C₁-C₆ alkyl.

In certain embodiments, X is CH₂, O, S, SO, SO₂ or NH. In certain embodiments, X is O, S, SO or SO₂. Preferably, X is O, and wherein the remaining variables are defined herein.

In certain embodiments, Y is CR¹⁰R¹¹, O, S, SO, SO₂, or NR¹⁰; wherein each R¹⁰ and R¹¹ independently is hydrogen or C₁-C₃ alkyl. In certain embodiments, Y is CR¹⁰R¹¹ wherein each R¹⁰ and R¹¹ independently is hydrogen or C₁-C₃ alkyl. Preferably, Y is CH₂, and wherein the remaining variables are defined herein.

In certain embodiments, t is 0. In certain embodiments, t is 1. In certain embodiments, t is 2. In certain embodiments, t is 3.

Preferably, CV¹V² is C═V, wherein V is O, and wherein the remaining variables are defined herein.

In certain embodiments, a compound of formula (III) is provided:

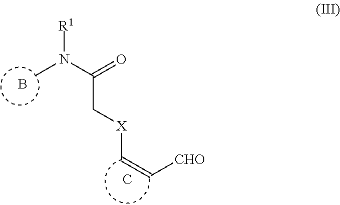

or a tautomer thereof, or a pharmaceutically acceptable salt of each thereof, wherein
ring B is C₆-C₁₀ aryl, C₃-C₈ cycloalkyl, a 5-10 membered heteroaryl containing up to 5 ring heteroatoms or a 4-10 membered heterocycle containing up to 5 ring heteroatoms, wherein the heteroatom is selected from the group consisting of O, N, S, and oxidized forms of N and S, wherein each of the aryl, heteroaryl, cycloalkyl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
X is O, S, SO or SO₂;
ring C is C₆-C₁₀ aryl or a 5-10 membered heteroaryl containing up to 5 ring heteroatoms, wherein the heteroatom is selected from the group consisting of O, N, S, and oxidized forms of N and S, each of which is optionally substituted with 1-4: halo, oxo, —OR², C₁-C₆ alkyl, and/or C₁-C₆ alkoxy, wherein the C₁-C₆ alkyl is optionally substituted with 1-5 halo, C₁-C₆ alkoxy and/or a 4-10 membered heterocycle containing up to 5 ring heteroatoms, wherein the heteroatom is selected from the group consisting of O, N, S, and oxidized forms of N and S;
R¹ is optionally substituted C₁-C₆ alkyl, C₂-C₆ alkynyl, C₂-C₆ alkynyl, C₆-C₁₀ aryl, a 5-10 membered heteroaryl, containing up to 5 ring heteroatoms wherein the heteroatom is selected from the group consisting of O, N, S, C₃-C₈ cycloalkyl or a 4-10 membered heterocycle containing up to 5 ring heteroatoms, wherein the heteroatom is selected from the group consisting of O, N, S; and
R² is hydrogen or a prodrug moiety R.

In certain embodiments, a compound of formula (IV) is provided:

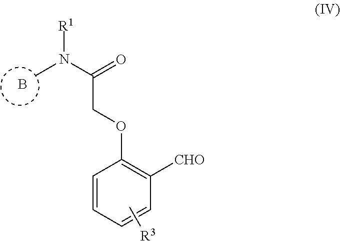

or a tautomer thereof, or a pharmaceutically acceptable salt of each thereof, wherein
ring B is C₆-C₁₀ aryl, C₃-C₈ cycloalkyl, a 5-10 membered heteroaryl containing up to 5 ring heteroatoms or a 4-10 membered heterocycle containing up to 5 ring heteroatoms, wherein the heteroatom is selected from the group consisting of O, N, S, and oxidized forms of N and S, wherein each of the aryl, heteroaryl, cycloalkyl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
R¹ is optionally substituted C₁-C₆ alkyl, C₂-C₆ alkynyl, or C₂-C₆ alkynyl, or is C₆-C₁₀ aryl, a 5-10 membered heteroaryl, containing up to 5 ring heteroatoms wherein the heteroatom is selected from the group consisting of O, N, S, C₃-C₈ cycloalkyl or a 4-10 membered heterocycle containing up to 5 ring heteroatoms, wherein the heteroatom is selected from the group consisting of O, N, S;
R³ is halo, oxo, C₁-C₆ alkyl and/or C₁-C₆ alkoxy; and
R⁴ is hydrogen or a prodrug moiety R.

In one embodiment, R³ is —OH.

In certain embodiments, ring B is

C₃-C₈ heteroaryl containing 1-3 heteroatoms, wherein the heteroaryl is optionally substituted with C₁-C₆ alkyl or C₁-C₆ alkoxy;
phenyl substituted with 1-3 halo, or
C₃-C₈ heterocyclyl containing 1-3 heteroatoms.

In certain embodiments, compounds of formulas (II), (III) and (IV) are provided, wherein

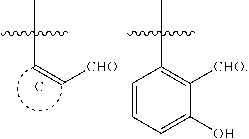

In certain embodiments, a compound is provided, wherein the compound is selected from the group consisting of:

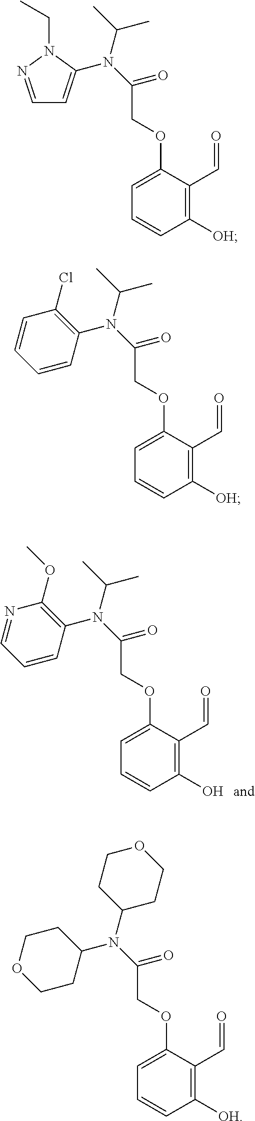 [SMILES: CCn1nccc1N(C(=O)COc1cccc(O)c1C=O)C(C)C ; CC(C)N(C(=O)COc1cccc(O)c1C=O)c1ccccc1Cl ; COc1ncccc1N(C(=O)COc1cccc(O)c1C=O)C(C)C ; O=Cc1c(O)cccc1OCC(=O)N(C1CCOCC1)C1CCOCC1]

or N oxides thereof, or a pharmaceutically acceptable salt of each thereof.

In certain aspects of the invention, a compound of formula (V) is provided:

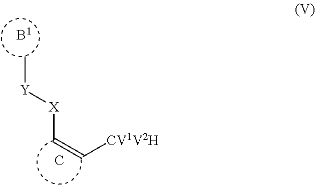

or a tautomer thereof, or a pharmaceutically acceptable salt of each thereof, wherein
ring B¹ is a 5-10 membered heteroaryl containing up to 5 ring heteroatoms or 4-10 membered heterocycle containing up to 5 ring heteroatoms, wherein the heteroatom is selected from the group consisting of O, N, S, and oxidized forms of N and S, wherein at least one of the heteroatoms or oxidized forms thereof is γ to the position where Y is attached to B¹, each of the heteroaryl is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
each X and Y is independently CR¹⁰R¹¹, O, S, SO, SO₂, or NR¹⁰; each R¹⁰ and R¹¹ independently is hydrogen or C₁-C₃ alkyl optionally substituted with 1-3 halo, OH, or C₁-C₆ alkoxy, or CR¹⁰R¹¹ is C═O, provided that if one of Y and Z is O, S, SO, SO₂, then the other is not CO, and Y and Z are both not heteroatoms or oxidized forms thereof;
ring C is C₆-C₁₀ aryl or a 5-10 membered heteroaryl containing up to 5 ring heteroatoms, wherein the heteroatom is selected from the group consisting of O, N, S, and oxidized forms of N and S, each of which is optionally substituted with 1-4: halo, oxo, —OR², C₁-C₆ alkyl, and/or C₁-C₆ alkoxy, wherein the C₁-C₆ alkyl is optionally substituted with 1-5 halo, C₁-C₆ alkoxy and/or a 4-10 membered heterocycle containing up to 5 ring heteroatoms, wherein the heteroatom is selected from the group consisting of O, N, S, and oxidized forms of N and S;
R² is hydrogen or a prodrug moiety R;
V¹ and V² independently are C₁-C₆ alkoxy; or V¹ and V² together with the carbon atom they are attached to form a ring of formula:

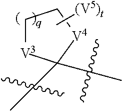

wherein each V³ and V⁴ are independently O, S, or NH, provided that when one of V³ and V⁴ is S, the other is NH, and provided that V³ and V⁴ are both not NH; q is 1 or 2; each V⁵ is independently C₁-C₆ alkyl optionally substituted with 1-3 OH groups or V⁵ is CO₂R⁶⁰, where each R⁶⁰ independently is C₁-C₆ alkyl or hydrogen; t is 0, 1, 2, or 4; or CV′V² is C═V, wherein V is O, NOR⁸⁰, or NNR⁸¹R⁸²;
R⁸⁰ is optionally substituted C₁-C₆ alkyl;
R⁸¹ and R⁸² independently are selected from the group consisting of hydrogen, optionally substituted C₁-C₆ alkyl, COR⁸³, or CO₂R⁸⁴;
R⁸³ is hydrogen or optionally substituted C₁-C₆ alkyl; and
R⁸⁴ is optionally substituted C₁-C₆ alkyl.

In certain embodiments, a compound of formula (VI) is provided:

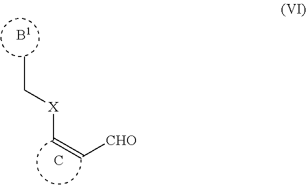

or a tautomer thereof, or a pharmaceutically acceptable salt of each thereof, wherein
ring B¹ is a 5-10 membered heteroaryl containing up to 5 ring heteroatoms or 4-10 membered heterocycle containing up to 5 ring heteroatoms, wherein the heteroatom is selected from the group consisting of O, N, S, and oxidized forms of N and S, wherein each of the heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
X is O, S, SO or SO₂;
ring C is C₆-C₁₀ aryl or a 5-10 membered heteroaryl containing up to 5 ring heteroatoms, wherein the heteroatom is selected from the group consisting of O, N, S, and oxidized forms of N and S, each of which is optionally substituted with 1-4: halo, oxo, —OR², C₁-C₆ alkyl, and/or C₁-C₆ alkoxy, wherein the C₁-C₆ alkyl is optionally substituted with 1-5 halo, C₁-C₆ alkoxy and/or a 4-10 membered heterocycle containing up to 5 ring heteroatoms, wherein the heteroatom is selected from the group consisting of O, N, S, and oxidized forms of N and S; and
R² is hydrogen or a prodrug moiety R.

In certain embodiments, a compound of formula (VII) is provided:

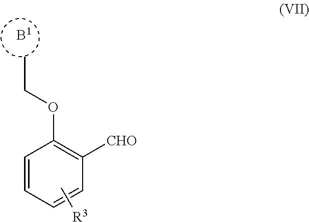

or a tautomer thereof, or a pharmaceutically acceptable salt of each thereof, wherein
ring B¹ is a 5-10 membered heteroaryl containing up to 5 ring heteroatoms or 4-10 membered heterocycle containing up to 5 ring heteroatoms, wherein the heteroatom is selected from the group consisting of O, N, S, and oxidized forms of N and S, wherein each of the aryl, heteroaryl, cycloalkyl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
R³ is halo, oxo, C₁-C₆ alkyl and/or C₁-C₆ alkoxy; and
R⁴ is hydrogen or a prodrug moiety R.

In one embodiment, R³ is —OH.

In certain embodiments, compounds of formulas (V), (VI) and (VII) are provided, wherein

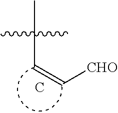

is

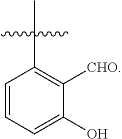 [SMILES: *c1cccc(O)c1C=O]

In one embodiment, ring B¹ is a 5-6 membered heterocycle containing a heteroatom selected from N, S, or O at least at a position which is 1,4 or γ from the 1-position noted above and wherein the heterocycle is substituted with a geminal ethylene (CH₂—CH₂).

In another embodiment, ring B¹ is a fused benzo ring which benzo ring is optionally substituted with oxo, C₁-C₆ alkyl, C₁-C₆ alkoxy or 1-5 halo.

In one embodiment, ring B¹ is selected from the group consisting of

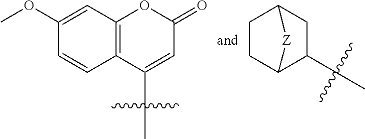

wherein Z is O or NR¹⁰ and R¹⁰ is hydrogen or optionally substituted C₁-C₆ alkyl.

In one embodiment, ring C is phenyl which is optionally substituted with 1-4: halo, oxo, —OR², C₁-C₆ alkyl and/or C₁-C₆ alkoxy.

In certain embodiment the following compound is provided:

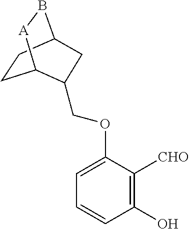

wherein each A and B independently are O, NR¹⁰, CH₂, or a bond, provided that A and B are both not O or a bond.

In certain embodiments, a compound is provided, wherein the compound is selected from the group consisting of:

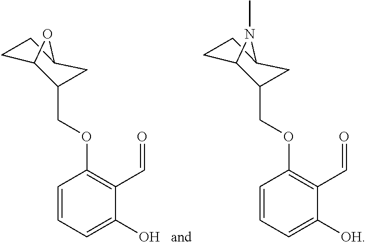 [SMILES: O=Cc1c(O)cccc1OCC1CC2CCC1O2 ; CN1C2CCC1C(COc1cccc(O)c1C=O)C2]

or N oxides thereof, or a pharmaceutically acceptable salt of each thereof.

In certain aspects of the invention, a compound of formula (VIII) is provided:

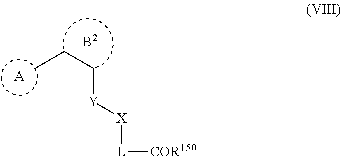

or a tautomer thereof, or a pharmaceutically acceptable salt of each thereof, wherein
ring A is a 5-10 membered heteroaryl, wherein the heteroatom is selected from the group consisting of O, N, S, and oxidized forms of N and S, wherein the heteroaryl is optionally substituted with 1-4: C₁-C₆ alkyl, wherein the C₁-C₆ alkyl is optionally substituted with 1-5 halo, C₁-C₆ alkoxy, and/or C₃-C₁₀ cycloalkyl;
ring B² is a 5-10 membered heteroaryl containing up to 5 ring heteroatoms, wherein the heteroatom is selected from the group consisting of O, N, S, and oxidized forms of N and S, wherein the heteroaryl is optionally substituted with with 1-4: C₁-C₆ alkyl, wherein the C₁-C₆ alkyl is optionally substituted with 1-5 halo, C₁-C₆ alkoxy, and/or C₃-C₁₀ cycloalkyl;
each X and Y is independently CR¹⁰R¹¹, O, S, SO, SO₂, or NR¹⁰; each R¹⁰ and R¹¹ independently is hydrogen or C₁-C₃ alkyl optionally substituted with 1-3 halo, OH, or C₁-C₆ alkoxy, or CR¹⁰R¹¹ is C═O, provided that if one of Y and Z is O, S, SO, SO₂, then the other is not CO, and Y and Z are both not heteroatoms or oxidized forms thereof;
wherein Y is a or β substituted relative to ring B;
L is joined with X and is a bond or is C₁-C₆ alkylene; and
R¹ is hydrogen, optionally substituted C₁-C₆ alkyl, C₂-C₆ alkynyl, or C₂-C₆ alkynyl, or is C₆-C₁₀ aryl, a 5-10 membered heteroaryl, containing up to 5 ring heteroatoms wherein the heteroatom is selected from the group consisting of O, N, S, C₃-C₈ cycloalkyl or a 4-10 membered heterocycle containing up to 5 ring heteroatoms, wherein the heteroatom is selected from the group consisting of O, N, S.

In certain embodiments, X is CH₂, O, S, SO, SO₂ or NH. In certain embodiments, X is O, S, SO or SO₂. Preferably, X is O, and wherein the remaining variables are defined herein.

In certain embodiments, Y is CR¹⁰R¹¹, O, S, SO, SO₂, or NR¹⁰; wherein each R¹⁰ and R¹¹ independently is hydrogen or C₁-C₃ alkyl. In certain embodiments, Y is CR¹⁰R¹¹ wherein each R¹⁰ and R¹¹ independently is hydrogen or C₁-C₃ alkyl. Preferably, Y is CH₂, and wherein the remaining variables are defined herein.

In certain embodiments, L is ethylene. In certain embodiments, L is methylene.

In certain embodiments, R¹ is hydrogen. In certain embodiments, R¹ is optionally substituted C₁-C₆ alkyl.

In certain embodiments, a compound of formula (IX) is provided:

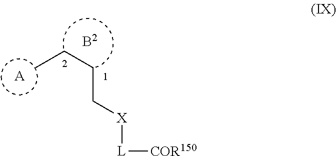

or a tautomer thereof, or a pharmaceutically acceptable salt of each thereof, wherein
ring A is a 5-10 membered heteroaryl, wherein the heteroatom is selected from the group consisting of O, N, S, and oxidized forms of N and S, wherein the heteroaryl is optionally substituted with 1-4: C₁-C₆ alkyl, wherein the C₁-C₆ alkyl is optionally substituted with 1-5 halo, C₁-C₆ alkoxy, and/or C₃-C₁₀ cycloalkyl;
ring B² is a 5-10 membered heteroaryl containing up to 5 ring heteroatoms, wherein the heteroatom is selected from the group consisting of O, N, S, and oxidized forms of N and S, wherein the heteroaryl is optionally substituted with with 1-4: C₁-C₆ alkyl, wherein the C₁-C₆ alkyl is optionally substituted with 1-5 halo, C₁-C₆ alkoxy, and/or C₃-C₁₀ cycloalkyl;
X is O, S, SO or SO₂; L is joined with X and is a bond or is C₁-C₆ alkylene; and
R¹⁵⁰ is hydrogen or optionally substituted C₁-C₆ alkyl.

It is contemplated that a compound of formula (VIII) is useful as an intermediate for making compounds that can modulate hemoglobin

In certain embodiments, a compound is provided, wherein the compound useful for making intermediates is selected from the group consisting of:

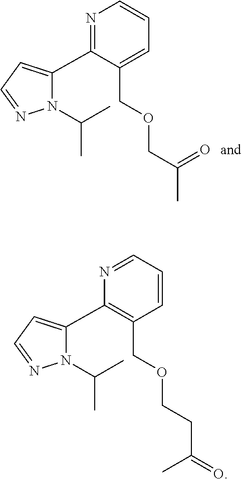 [SMILES: CC(=O)COCc1cccnc1-c1ccnn1C(C)C ; CC(=O)CCOCc1cccnc1-c1ccnn1C(C)C]

### Prodrug Moiety

In one aspect, the prodrug moiety in the compounds of invention is R. In certain embodiments, R is a phosphate or a diphosphate containing moiety, or another promoiety or prodrug moiety. Preferably the prodrug moiety imparts at least a 2 fold, more preferably a 4 fold, enhanced solubility and/or bioavailability to the active moiety (where R is hydrogen), and more preferably is hydrolyzed in vivo. The promoieties are structurally and functionally defined herein.

In one embodiments, R is —COR⁹⁰, CO₂R⁹¹, or CONR⁹²R⁹³ wherein

R⁹⁰ and R⁹¹ independently are C₁-C₆ alkyl, C₃-C₈ cycloalkyl, 4-9 membered heterocycle, or a 5-10 membered heteroaryl, each containing at least 1 basic nitrogen moiety; and
R⁹² and R⁹³ independently are C₁-C₆ alkyl; C₃-C₈ cycloalkyl, 4-9 membered heterocycle, or a 5-10 membered heteroaryl, each containing at least 1 basic nitrogen moiety; or R⁹² and R⁹³ together with the nitrogen atom they are bonded to for a 4-9 member heterocycle substituted with at least 1 amino, C₁-C₆ alkyl amino, or di C₁-C₆ alkylamino group.

In certain embodiments, R is —C(O)R³¹, C(O)OR³¹, or CON(R¹³)₂,

each R³¹ is independently a C₁-C₆ alkyl; C₃-C₈ cycloalkyl, 4-9 membered heterocycle, or a 5-10 membered heteroaryl, containing at least 1 basic nitrogen moiety; and

each R¹³ independently is C₁-C₆ alkyl; C₃-C₈ cycloalkyl, 4-9 membered heterocycle, or a 5-10 membered heteroaryl, containing at least 1 basic nitrogen moiety; or both R¹³ together with the nitrogen atom they are bonded to for a 4-9 member heterocycle substituted with at least 1 amino, C₁-C₆ alkyl amino, or di C₁-C₆ alkylamino group.

In one aspect, R is C(O)OR³¹, C(S)OR³¹, C(O)SR³¹ or COR³¹, wherein R³¹ is as defined herein.

In one embodiment, R¹¹ is a group of the formula (CR³²R³³)ₑNR³⁴R³⁵, wherein

each R³² and R³³ is independently H, a C₁-C₈ alkyl, C₃-C₉ heterocyclyl, C₃-C₈ cycloalkyl, C₆-C₁₀ aryl, C₃-C₉ heteroaryl or R³² and R³³ together with the carbon atom they are bond to form a C₃-C₈ cycloalkyl, C₆-C₁₀ aryl, C₃-C₉ heterocyclyl or C₃-C₉ heteroaryl ring system, or 2 adjacent R³² moieties or 2 adjacent R³³ moieties together with the carbon atom they are bond to form a C₃-C₈ cycloalkyl, C₆-C₁₀ aryl, C₃-C₉ heterocyclyl or C₃-C₉ heteroaryl ring system;

each R³⁴ and R³⁵ is a C₁-C₈ alkyl, C₃-C₉ heterocyclyl, C₃-C₈ cycloalkyl, or R³⁴ and R³⁵ together with the nitrogen atom they are bond to form a C₃-C₈ cycloalkyl or C₃-C₉ heterocyclyl ring system;

each heterocyclic and heteroaryl ring system is optionally substituted with C₁-C₃ alkyl, —OH, amino and carboxyl groups; and

e is an integer of from 1 to 4.

In some less preferred embodiments R³⁴ and R³⁵ can be hydrogen.

In one embodiment, the subscript e is preferably 2 and each R³² and R³³ is preferably independently selected from the group, H, CH₃, and a member in which R³² and R³³ are joined together to form a cyclopropyl, cyclobutyl, cyclopentyl, cyclohexyl, or 1,1-dioxo-hexahydro-1Δ⁶-thiopyran-4-yl or tetrahydropyran-4-yl group.

With regard to the prodrug group, preferred embodiments are compounds wherein NR³⁴R³⁵ is morpholino.

In one embodiment, R is:

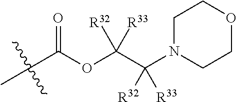

wherein

each R³² and R³³ is independently H, C₁-C₈ alkyl, or optionally, if both present on the same substituent, may be joined together to form a C₃-C₈ cycloalkyl, C₆-C₁₀ aryl, C₃-C₉ heterocyclyl or C₃-C₉ heteroaryl ring system.

Within this embodiment, each R³² and R³³ is independently, H, CH₃, or are joined together to form a cyclopropyl, cyclopbutyl, cyclopentyl, cyclohexyl, 1,1-dioxo-hexahydro-1λ⁶-thiopyran-4-yl or tetrahydropyran-4-yl group.

In a preferred embodiment, linkage of the prodrug moiety to the rest of the active molecule is stable enough so that the serum half life of the prodrug is from about 8 to about 24 hours.

In an embodiment of the invention, the prodrug moiety comprises a tertiary amine having a pKa near the physiological pH of 7.5. Any amines having a pKa within 1 unit of 7.5 are suitable alternatives amines for this purpose. The amine may be provided by the amine of a morpholino group. This pKa range of 6.5 to 8.5 allows for significant concentrations of the basic neutral amine to be present in the mildly alkaline small intestine. The basic, neutral form of the amine prodrug is lipophilic and is absorbed through the wall of the small intestine into the blood. Following absorption into the bloodstream, the prodrug moiety is cleaved by esterases which are naturally present in the serum to release an active compound.

Examples of R include, without limitation:

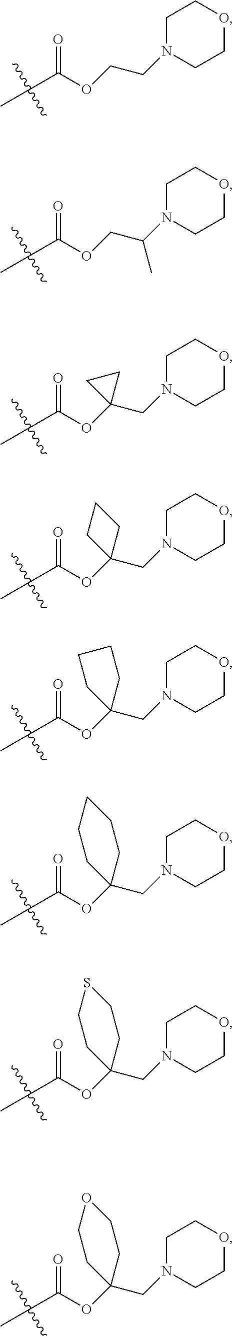

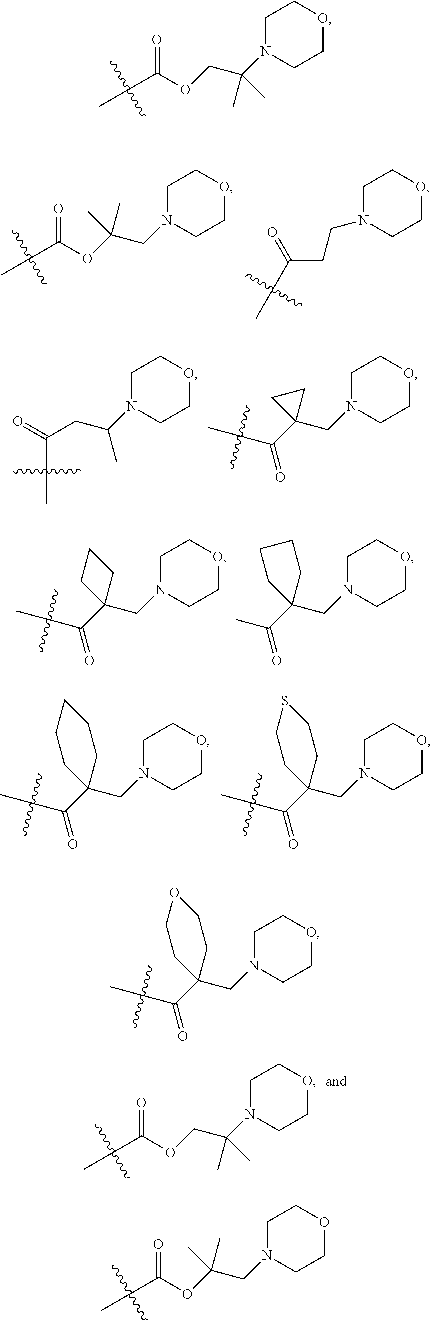

In another embodiment, R is as tabulated below:

| R | m | R³⁴ | R³⁵ | NR³⁴R³⁵ |
|---|---|---|---|---|
| C(O)(CH₂)ₘNR³⁴R³⁵ | 1 | Me | Me | |
| C(O)(CH₂)ₘNR³⁴R³⁵ | 2 | Me | Me | |
| C(O)(CH₂)ₘNR³⁴R³⁵ | 3 | Me | Me | |
| C(O)(CH₂)ₘNR³⁴R³⁵ | 4 | Me | Me | |
| C(O)(CH₂)ₘNR³⁴R³⁵ | 1 | | | 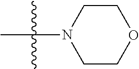 [SMILES: *N1CCOCC1] |
| C(O)(CH₂)ₘNR³⁴R³⁵ | 2 | | | 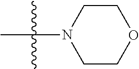 [SMILES: *N1CCOCC1] |
| C(O)(CH₂)ₘNR³⁴R³⁵ | 3 | | | 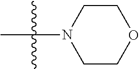 [SMILES: *N1CCOCC1] |
| C(O)(CH₂)ₘNR³⁴R³⁵ | 4 | | | 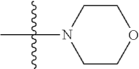 [SMILES: *N1CCOCC1] |
| C(O)O(CH₂)ₘNR³⁴R³⁵ | 2 | Me | Me | |
| C(O)O(CH₂)ₘNR³⁴R³⁵ | 3 | Me | Me | |
| C(O)O(CH₂)ₘNR³⁴R³⁵ | 4 | Me | Me | |
| C(O)O(CH₂)ₘNR³⁴R³⁵ | 2 | | | 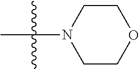 [SMILES: *N1CCOCC1] |
| C(O)O(CH₂)ₘNR³⁴R³⁵ | 3 | | | 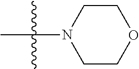 [SMILES: *N1CCOCC1] |
| C(O)O(CH₂)ₘNR³⁴R³⁵ | 4 | | | 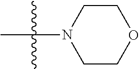 [SMILES: *N1CCOCC1] |
| P(O)(OH)₂ | | | | |

an N oxide thereof, or a pharmaceutically acceptable salt of each thereof.

In another aspect, R is,

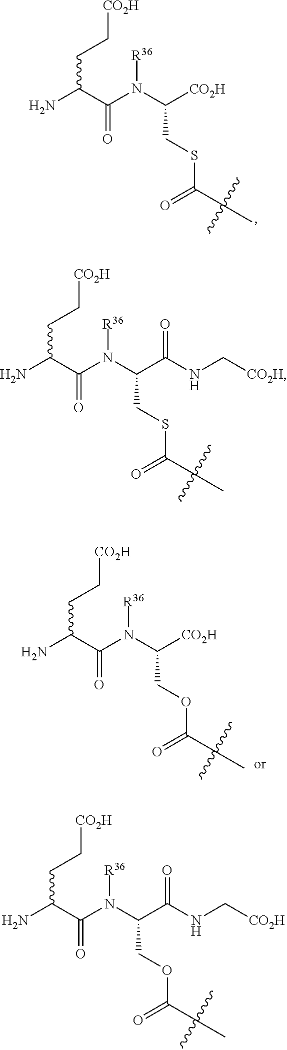

wherein

R³⁶ is lower alkyl (e.g. C₁-C₆ alkyl).

In yet another aspect, R is:

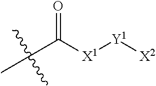

wherein X % Y¹ and X² are as defined herein.

In one embodiment, X¹ is selected from the group consisting of O, S and NR³⁷ wherein R³⁷ is hydrogen or C₁-C₆ alkyl;

Y¹ is —C(R³⁸)₂ or a sugar moiety, wherein each R³⁸ is independently hydrogen or C₁-C₆ alkyl, C₃-C₈ cycloalkyl, C₃-C₉ heterocyclyl, C₆-C₁₀ aryl, or C₃-C₉ heteroaryl;

X² is selected from the group consisting of halogen, C₁-C₆ alkoxy, diacylglycerol, amino, C₁-C₆ alkylamino, C₁-C₆ dialkylamino, C₁-C₆ alkylthio, a PEG moiety, a bile acid moiety, a sugar moiety, an amino acid moiety, a di- or tri-peptide, a PEG carboxylic acid, and —U—V wherein

U is O or S; and

V is selected from the group consisting of C₁-C₆ alkyl, C₃-C₈ cycloalkyl, C₃-C₉ heterocyclyl, C₆-C₁₀ aryl, C₃-C₉ heteroaryl, C(W²)X³, PO(X³)₂, and SO₂X³;

wherein W² is O or NR³⁹

wherein R³⁹ is hydrogen or C₁-C₆ alkyl, C₃-C₈ cycloalkyl, C₃-C₉ hetrocyclyl, C₆-C₁₀ aryl, or C₃-C₉ heteroaryl; and

each X³ is independently amino, hydroxyl, mercapto, C₁-C₆ alkyl, heteroalkyl, cycloalkyl, hetrocyclyl, aryl, or heteroaryl, C₁-C₆ alkoxy, C₁-C₆ alkylamino, C₁-C₆ dialkylamino, C₁-C₆ alkylthio, a bile acid based alkoxy group, a sugar moiety, a PEG moiety, and —O—CH₂—CH(OR⁴⁰)CH₂X⁴R⁴⁰,

wherein:

X⁴ is selected from the group consisting of O, S, S═O, and SO₂; and

each R⁴⁰ is independently C₁₀-C₂₂ alkyl, C₃-C₈ cycloalkyl, C₃-C₉ heterocyclyl, C₆-C₁₀ aryl, or C₃-C₉ heteroaryl, C₁-C₈ alkylene, or C₁-C₈ heteroalkylene.

Each heterocyclic and heteroaryl ring system is optionally substituted with C₁-C₃ alkyl, —OH, amino and carboxyl groups.

In one embodiment, the present invention utilizes the following Y¹ groups: CH₂, CHMe, CH(isopropyl), CH(tertiarybutyl), C(Me)₂, C(Et)₂, C(isopropyl)₂, and C(propyl)₂.

In another embodiment, the present invention utilizes the following X² groups:

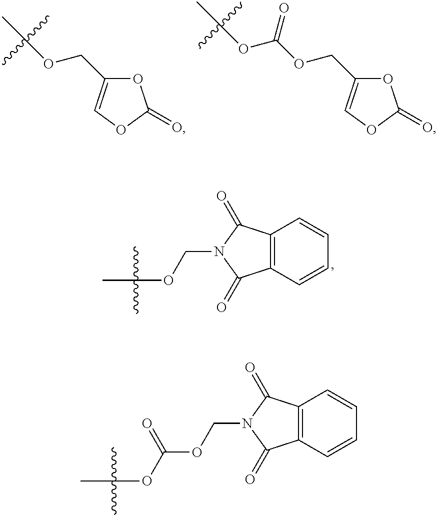 [SMILES: *OCc1coc(=O)o1 ; *OC(=O)OCc1coc(=O)o1 ; *OCN1C(=O)c2ccccc2C1=O ; *OC(=O)OCN1C(=O)c2ccccc2C1=O]

—OMe, —OEt, —O-isopropyl, O-isobutyl, O-tertiarybutyl, —O—COMe, —O—C(═O)(isopropyl), —O—C(═O)(isobutyl), —O—C(═O)(tertiarybutyl), —O—C(═O)—NMe₂, —O—C(═O)—NHMe, —O—C(═O)—NH₂, —O—C(═O)—N(H)—CH(R⁴¹)—CO₂Et wherein R⁴¹ is a side chain C₁-C₆ alkyl, or C₃-C₉ heterocyclyl group selected from the side chain groups present in essential amino acids; —O—P(═O)(OMe)₂, —O—P(═O)(O-isopropyl)₂, and —O—P(═O)(O-isobutyl)₂. Each heterocyclic is optionally substituted with one or more, preferably, 1-3, C₁-C₃ alkyl, —OH, amino and/or carboxyl groups.

In another embodiment, R is:

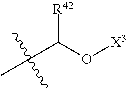

wherein

X³ is independently C₁-C₆ alkyl, C₃-C₈ cycloalkyl, C₃-C₉ heterocyclyl, C₆-C₁₀ aryl, or C₃-C₉ heteroaryl; and

R⁴² is independently hydrogen or C₁-C₆ alkyl, C₃-C₈ cycloalkyl, C₃-C₉ heterocyclyl, C₆-C₁₀ aryl, or C₃-C₉ heteroaryl.

Each heterocyclic is optionally substituted with one or more, preferably, 1-3, C₁-C₃ alkyl, —OH, amino and/or carboxyl groups.

In one embodiment, R is:

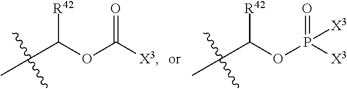

wherein

each X³ is independently amino, hydroxyl, mercapto, C₁-C₆ alkyl, C₃-C₈ cycloalkyl, C₃-C₉ heterocyclyl, C₆-C₁₀ aryl, or C₃-C₉ heteroaryl, C₁-C₆ alkoxy, C₁-C₆ alkylamino, C₁-C₆ dialkylamino, C₁-C₆ alkylthio, a bile acid based alkoxy group, a sugar moiety, a PEG moiety, and —O—CH₂—CH(OR⁴⁰)CH₂X⁴R⁴⁰,

wherein:

X⁴ is selected from the group consisting of O, S, S═O, and SO₂; and

each R⁴⁰ is independently C₁₀-C₂₂ alkyl, C₃-C₈ cycloalkyl, C₃-C₉ heterocyclyl, C₆-C₁₀ aryl, C₃-C₉ heteroaryl, C₁-C₈ alkylene, or C₁-C₈ heteroalkylene; and

R⁴² is independently hydrogen or C₁-C₆ alkyl, C₃-C₈ cycloalkyl, C₃-C₉ heterocyclyl, C₆-C₁₀ aryl, or C₃-C₉ heteroaryl.

In some embodiments, R⁴² is independently hydrogen or C₁-C₆ alkyl, C₃-C₈ cycloalkyl, C₃-C₉ heterocyclyl, C₆-C₁₀ aryl, or C₃-C₉ heteroaryl; and each X³ independently is C₁-C₆ alkyl, C₃-C₈ cycloalkyl, C₃-C₉ heterocyclyl, C₆-C₁₀ aryl, or C₃-C₉ heteroaryl, C₁-C₆ alkoxy, C₁-C₆ alkylamino, C₁-C₆ dialkylamino, or C₁-C₆ alkylthio.

In certain embodiments, R is represented by the following structures:

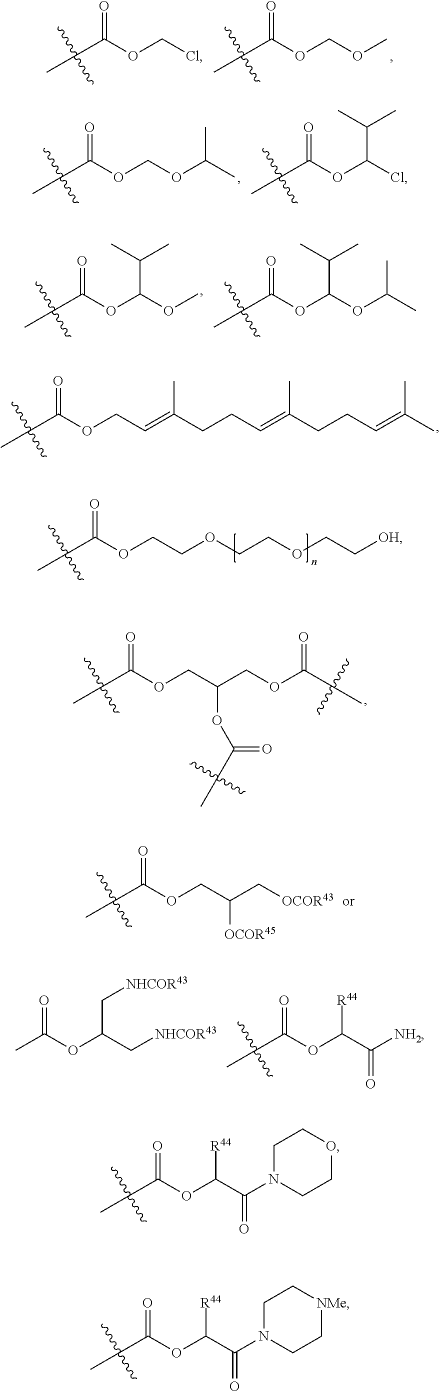

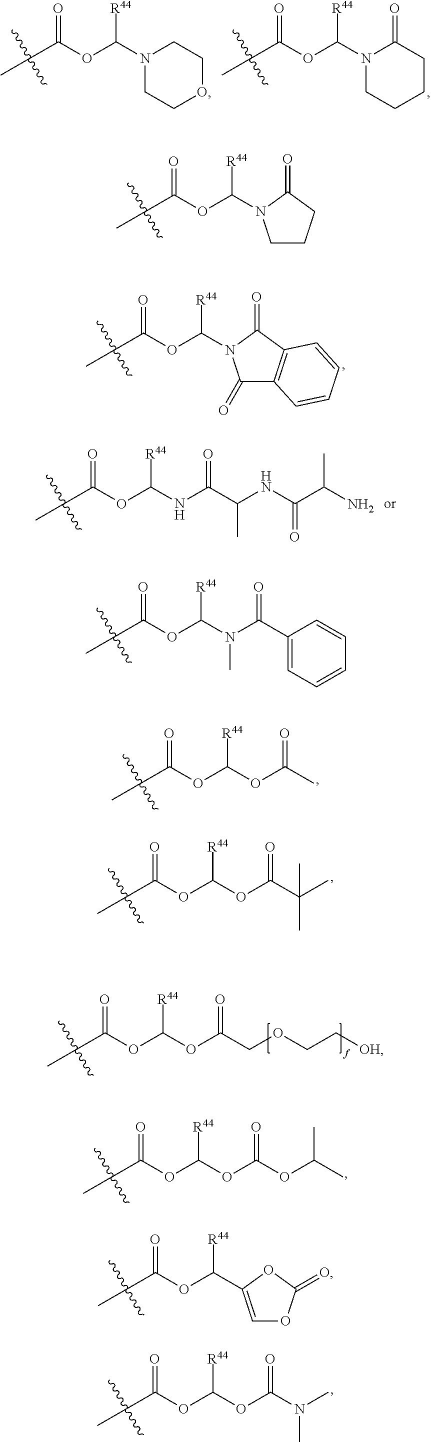

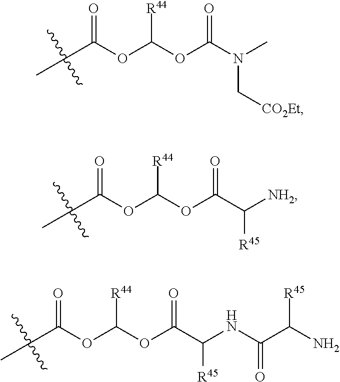

wherein, in the above examples, R⁴³ is C₁₀-C₂₂ alkyl or alkylene, R⁴⁴ is H or C₁-C₆ alkyl and R⁴⁵ represents side chain alkyl groups present in naturally occurring alpha amino acids;

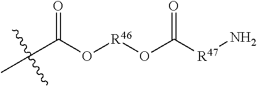

wherein R⁴⁶ is (CH₂)ₙ, f=2-4, and CO—R⁴⁷—NH₂ represents an aminoacyl group; or

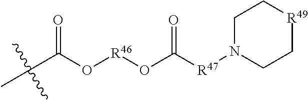

wherein R⁴⁶ is (CH₂)ₙ, n=2-4, R⁴⁷ is (CH₂)ₙ, n=1-3 and R⁴⁹ is O or NMe.

In one embodiment, R is:

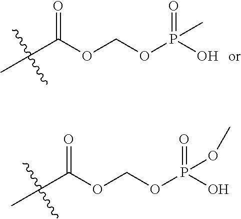

In one aspect, R is —C(R²⁰⁰R²⁰¹)O(R²⁰²R²⁰³)P(O)OR²⁰⁴NR²⁰⁵R²⁰⁶, wherein each R²⁰⁰, R²⁰¹, R²⁰², R²⁰³, R²⁰⁴R²⁰⁵ and R²⁰⁶ is independently H, a C₁-C₈ alkyl, C₃-C₉ heterocyclyl, C₃-C₈ cycloalkyl, C₆-C₁₀ aryl, C₃-C₉ heteroaryl, wherein each alkyl, heterocyclyl, cycloalkyl, aryl, and heteroaryl is optionally substituted.

In some embodiments, R is —CH(R²⁰¹)OCH₂P(O)OR²⁰⁴NHR²⁰⁶, wherein R²⁰¹ is C₁-C₈ alkyl, R²⁰⁴ is phenyl, optionally substituted. In one embodiment, R²⁰⁶ is —CHR²⁰⁷C(O)OR²⁰⁸ wherein R²⁰⁷ is selected from the group consisting of the naturally occurring amino acid side chains and —CO₂H esters thereof and R²⁰⁸ is C₁-C₈ alkyl. In one embodiment, R²⁰⁶ is C₁-C₆ alkyl, optionally substituted with 1-3, CO₂H, SH, NH₂, C₆-C₁₀ aryl, and C₂-C₁₀ heteroaryl.

In one embodiment, R is:

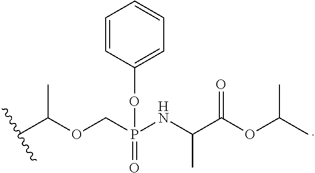 [SMILES: *C(C)OCP(=O)(NC(C)C(=O)OC(C)C)Oc1ccccc1]

In one embodiment, R is —P(═O)(OH)₂ or a mono or bis salt thereof.

In one embodiment, R is:

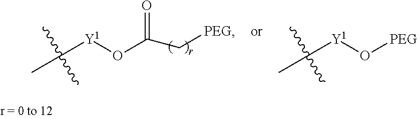

wherein Y¹ is —C(R³⁸)₂, wherein each R³⁸ is independently hydrogen or C₁-C₆ alkyl, C₃-C₈ cycloalkyl, C₃-C₉ heterocyclyl, C₆-C₁₀ aryl, or C₃-C₉ heteroaryl.

Various polyethylene glycol (PEG) moieties and synthetic methods related to them that can be used or adapted to make compounds of the invention are described in U.S. Pat. Nos. 6,608,076; 6,395,266; 6,194,580; 6,153,655; 6,127,355; 6,111,107; 5,965,566; 5,880,131; 5,840,900; 6,011,042 and 5,681,567.

In one embodiment, R is

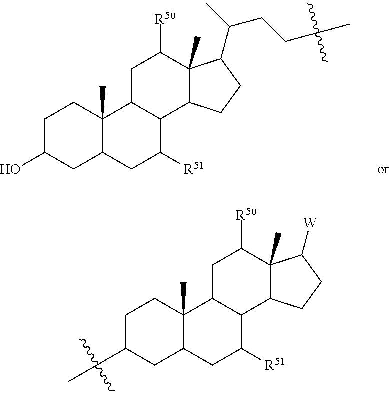

wherein

R⁵⁰ is —OH or hydrogen;

R⁵¹ is —OH, or hydrogen;

W is —CH(CH₃)W¹;

wherein W¹ is a substituted C₁-C₈ alkyl group containing a moiety which is optionally negatively charged at physiological pH,

said moiety is selected from the group consisting of CO₂H, SO₃H, SO₂H, —P(O)(OR⁵²)(OH), —OP(O)(OR⁵²)(OH), and OSO₃H,

wherein R⁵² is C₁-C₆ alkyl, C₃-C₈ cycloalkyl, C₃-C₉ heterocyclyl, C₆-C₁₀ aryl, or C₃-C₉ heteroaryl.

Each heterocyclic and heteroaryl ring system is optionally substituted with one or more, preferably 1-3, C₁-C₃ alkyl, —OH, amino and/or carboxyl groups.

In one embodiment, R is:

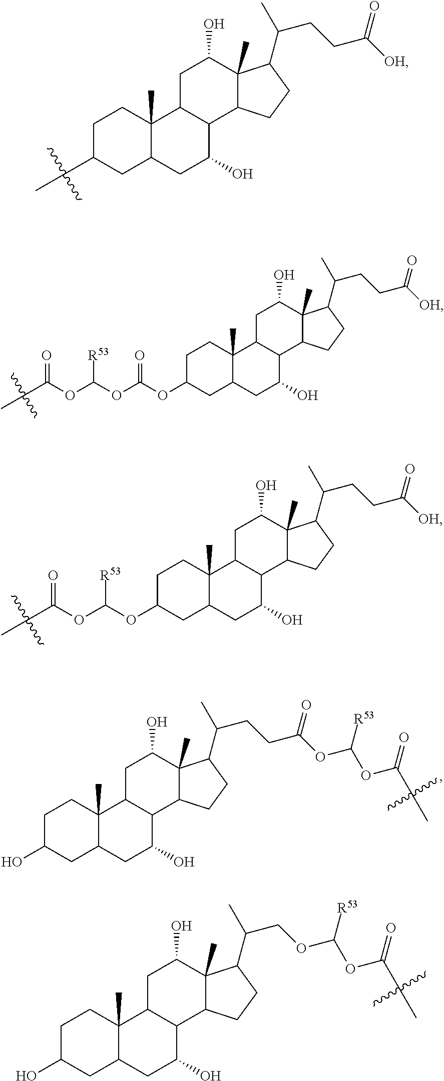

wherein R⁵³ is H or C₁-C₆ alkyl.

In another aspect, R is SO₃H.

In another aspect, R comprises a cleavable linker, wherein the term “cleavable linker” refers to a linker which has a short half life in vivo. The breakdown of the linker Z in a compound releases or generates the active compound. In one embodiment, the cleavable linker has a half life of less than ten hours. In one embodiment, the cleavable linker has a half life of less than an hour. In one embodiment, the half life of the cleavable linker is between one and fifteen minutes. In one embodiment, the cleavable linker has at least one connection with the structure: C*—C(═X*)X*—C* wherein C* is a substituted or unsubstituted methylene group, and X* is S or O. In one embodiment, the cleavable linker has at least one C*—C(═O)O—C* connection. In one embodiment, the cleavable linker has at least one C*—C(═O)S—C* connection. In one embodiment, the cleavable linker has at least one —C(═O)N*—C*—SO₂—N*-connection, wherein N* is —NH— or C₁-C₆ alkylamino. In one embodiment, the cleavable linker is hydrolyzed by an esterase enzyme.

In one embodiment, the linker is a self-immolating linker, such as that disclosed in U.S. patent publication 2002/0147138, to Firestone; PCT Appl. No. US05/08161 and PCT Pub. No. 2004/087075. In another embodiment, the linker is a substrate for enzymes. See generally Rooseboom et al., 2004, Pharmacol. Rev. 56:53-102.

### Pharmaceutical Compositions

In further aspects of the invention, a composition is provided comprising any of the compounds described herein, and at least a pharmaceutically acceptable excipient.

In another aspect, this invention provides a composition comprising any of the compounds described herein, and a pharmaceutically acceptable excipient.

Such compositions can be formulated for different routes of administration. Although compositions suitable for oral delivery will probably be used most frequently, other routes that may be used include transdermal, intravenous, intraarterial, pulmonary, rectal, nasal, vaginal, lingual, intramuscular, intraperitoneal, intracutaneous, intracranial, and subcutaneous routes. Suitable dosage forms for administering any of the compounds described herein include tablets, capsules, pills, powders, aerosols, suppositories, parenterals, and oral liquids, including suspensions, solutions and emulsions. Sustained release dosage forms may also be used, for example, in a transdermal patch form. All dosage forms may be prepared using methods that are standard in the art (see e.g., Remington's Pharmaceutical Sciences, 16th ed., A. Oslo editor, Easton Pa. 1980).

Pharmaceutically acceptable excipients are non-toxic, aid administration, and do not adversely affect the therapeutic benefit of the compound of this invention. Such excipients may be any solid, liquid, semi-solid or, in the case of an aerosol composition, gaseous excipient that is generally available to one of skill in the art. Pharmaceutical compositions in accordance with the invention are prepared by conventional means using methods known in the art.

The compositions disclosed herein may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerin and the like.

Solid pharmaceutical excipients include starch, cellulose, hydroxypropyl cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. In certain embodiments, the compositions provided herein comprises one or more of α-tocopherol, gum arabic, and/or hydroxypropyl cellulose.

In one embodiment, this invention provides sustained release formulations such as drug depots or patches comprising an effective amount of a compound provided herein. In another embodiment, the patch further comprises gum Arabic or hydroxypropyl cellulose separately or in combination, in the presence of alpha-tocopherol. Preferably, the hydroxypropyl cellulose has an average MW of from 10,000 to 100,000. In a more preferred embodiment, the hydroxypropyl cellulose has an average MW of from 5,000 to 50,000.

Compounds and pharmaceutical compositions of this invention maybe used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the compound of this invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

### Methods of Treatment

In aspects of the invention, a method is provided for increasing tissue and/or cellular oxygenation, the method comprising administering to a subject in need thereof a therapeutically effective amount of any of the compounds or compositions described herein.

In aspects of the invention, a method is provided for increasing oxygen affinity of hemoglobin S in a subject, the method comprising administering to a subject in need thereof a therapeutically effective amount of any of the compounds or compositions described herein.

In aspects of the invention, a method is provided for treating a condition associated with oxygen deficiency, the method comprising administering to a subject in need thereof a therapeutically effective amount of any of the compounds or compositions described herein.

In further aspects of the invention, a method is provided for treating oxygen deficiency associated with sickle cell anemia, the method comprising administering to a subject in need thereof a therapeutically effective amount of any of the compounds or compositions described herein.

In further aspects of the invention, a method is provided for treating sickle cell disease, the method comprising administering to a subject in need thereof a therapeutically effective amount of a compound of any of the compounds or compositions described herein. In still further aspects of the invention, a method is provided for treating cancer, a pulmonary disorder, stroke, high altitude sickness, an ulcer, a pressure sore, Alzheimer's disease, acute respiratory disease syndrome, and a wound, the method comprising administering to a subject in need thereof a therapeutically effective amount of a compound of any of the compounds or compositions described herein.

### Synthetic Methods

Certain methods for making the compounds described herein are also provided. The reactions are preferably carried out in a suitable inert solvent that will be apparent to the skilled artisan upon reading this disclosure, for a sufficient period of time to ensure substantial completion of the reaction as observed by thin layer chromatography, ¹H-NMR, etc. If needed to speed up the reaction, the reaction mixture can be heated, as is well known to the skilled artisan. The final and the intermediate compounds are purified, if necessary, by various art known methods such as crystallization, precipitation, column chromatography, and the likes, as will be apparent to the skilled artisan upon reading this disclosure.

An illustrative and non-limiting method for synthesizing a compound of formula (I), is schematically shown below.

### General Synthetic Schemes

### Prodrug Synthesis

Syntheses of the ester prodrugs start with the free carboxylic acid bearing the tertiary amine. The free acid is activated for ester formation in an aprotic solvent and then reacted with a free alcohol group in the presence of an inert base, such as triethyl amine, to provide the ester prodrug. Activating conditions for the carboxylic acid include forming the acid chloride using oxalyl chloride or thionyl chloride in an aprotic solvent, optionally with a catalytic amount of dimethyl formamide, followed by evaporation. Examples of aprotic solvents, include, but are not limited to methylene chloride, tetrahydrofuran, and the like. Alternatively, activations can be performed in situ by using reagents such as BOP (benzotriazol-1-yloxytris(dimethylamino) phosphonium hexafluorolphosphate, and the like (see Nagy et al., 1993, Proc. Natl. Acad. Sci. USA 90:6373-6376) followed by reaction with the free alcohol. Isolation of the ester products can be affected by extraction with an organic solvent, such as ethyl acetate or methylene chloride, against a mildly acidic aqueous solution; followed by base treatment of the acidic aqueous phase so as to render it basic; followed by extraction with an organic solvent, for example ethyl acetate or methylene chroride; evaporation of the organic solvent layer; and recrystalization from a solvent, such as ethanol. Optionally, the solvent can be acidified with an acid, such as HCl or acetic acid to provide a pharmaceutically acceptable salt thereof. Alternatively the crude reaction can be passed over an ion exchange column bearing sulfonic acid groups in the protonated form, washed with deionized water, and eluted with aqueous ammonia; followed by evaporation.

Suitable free acids bearing the tertiary amine are commercially available, such as 2-(N-morpholino)-propionic acid, N,N-dimethyl-beta-alanine, and the like. Non-commercial acids can be synthesized in straightforward manner via standard literature procedures.

Carbonate and carbamate prodrugs can be prepared in an analogous way. For example, amino alcohols and diamines can be activated using activating agents such as phosgene or carbonyl diimidazole, to provide an activated carbonates, which in turn can react with the alcohol and/or the phenolic hydroxy group on the compounds utilized herein to provide carbonate and carbamate prodrugs.

Various protecting groups and synthetic methods related to them that can be used or adapted to make compounds of the invention can be adapted from the references Testa et al., Hydrolysis in Drug and Prodrug Metabolism, June 2003, Wiley-VCH, Zurich, 419-534 and Beaumont et al., Curr. Drug Metab. 2003, 4:461-85.

Scheme A below provides a method of synthesizing an acyloxymethyl version of a prodrug by adapting a method from the reference Sobolev et al., 2002, J. Org. Chem. 67:401-410.

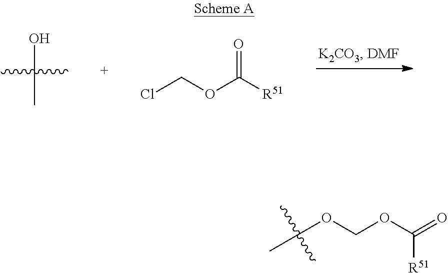

wherein R⁵¹ is C₁-C₆ alkyl.

Scheme B below provides a method for synthesizing a phosphonooxymethyl version of a prodrug by adapting a method from Mantyla et al., 2004, J. Med. Chem. 47:188-195.

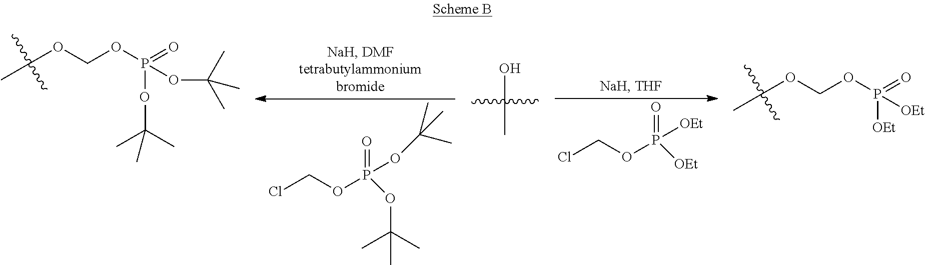

Scheme C below provides a method of synthesizing an alkyloxymethyl version of a prodrug

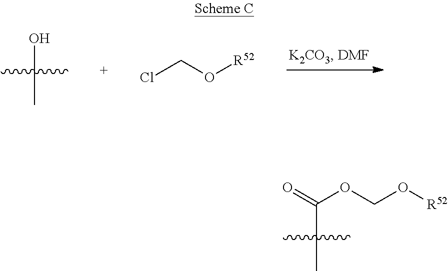

wherein R⁵² is C₁-C₆ alkyl, C₃-C₈ cycloalkyl, C₃-C₉ heterocyclyl, C₆-C₁₀ aryl, or C₃-C₉ heteroaryl.

### Examples

In the examples below as well as throughout the application, the following abbreviations have the following meanings. If not defined, the terms have their generally accepted meanings.
° C.=degrees Celsius
RT=Room temperature
min=minute(s)
h=hour(s)
μL=Microliter
mL=Milliliter
mmol=Millimole
eq=Equivalent
mg=Milligram
ppm=Parts per million
atm=Atmospheric pressure
MS=Mass spectrometry
LC-MS=Liquid chromatography-mass spectrometry
HPLC=High performance liquid chromatography
NMR=Nuclear magnetic resonance
Sat./sat. Saturated
DMF=N, N-Dimethylformamide
DCM=Dichloromethane
LAH/LiAlH₄=Lithium aluminum hydride
THF=Tetrahydrofuran
DIBAL=Diisobutylaluminium hydride
DIAD=Diisopropyl azodicarboxylate
MOM Methoxymethyl ether

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Experimental Procedures for Examples:

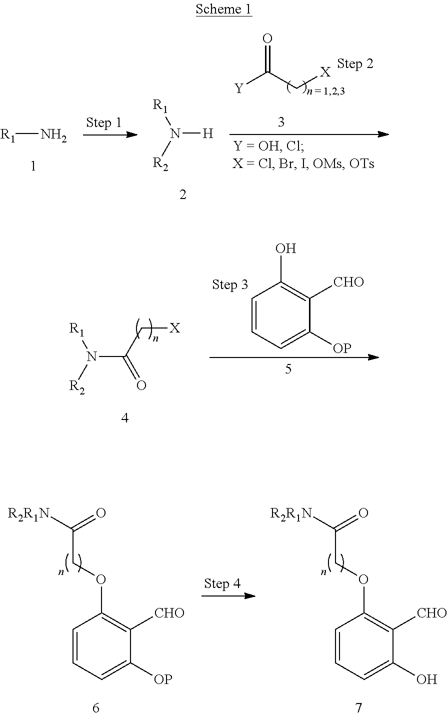

Compound 7 can be synthesized using general organic transformations described in Scheme 1. Amide coupling of tertiary amine 2 with w-halo acid derivative 3 yields amide 4 (step 2), which is transformed via 0-phenol alkylation with 2-hydroxybenzaldehyde 5 to key intermediate 6 (step 3). Removal of protecting group from 6 (such as MOM ether, P=MOM) under appropriate conditions (acidic conditions for removing MOM group) provides the final product 7 (step 4).

### Experimental Procedures for Examples:

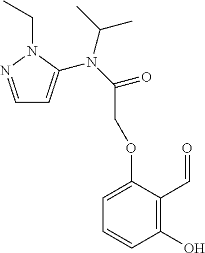 [SMILES: CCn1nccc1N(C(=O)COc1cccc(O)c1C=O)C(C)C]

### GBT001061 Preparation of N-(1-ethyl-1H-pyrazol-5-yl)-2-(2-formyl-3-hydroxyphenoxy)-N-isopropylacetamide

### Step 1

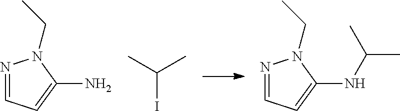

1-Ethyl-1H-pyrazol-5-amine (0.7 g, 6.3 mmol) was dissolved in THF (5 ml). Sodium hydride (0.5 g, 60% dispersion in mineral oil, 12.6 mmol) was added and the mixture stirred for 10 m. 2-iodopropane (0.82 g, 8.2 mmol) was added and the mixture stirred for 16 h. Water (50 ml) and ethyl acetate (100 ml) were added. The phases were separated and the aqueous phase was extracted with ethyl acetate (2×75 ml). The combined organic phases were washed with a saturated aqueous sodium chloride solution (30 ml) and then dried over sodium sulfate. After evaporation, the residue was purified by silica gel chromatography (5-50% ethyl acetate/hexanes) to give 0.49 g (50%) of 1-ethyl-N-isopropyl-1H-pyrazol-5-amine as a yellowish oil. MS (ESI) m/z 154 [M+H]⁺.

### Step 2

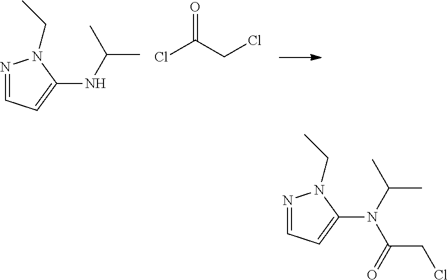

1-Ethyl-N-isopropyl-1H-pyrazol-5-amine (0.49 g, 3.2 mmol) was dissolved in dichloromethane (11 ml) and stirred in an ice bath. N,N-diisopropylethylamine (1.14 ml, 6.4 mmol) was added followed by slow addition of chloroacetyl chloride (0.51 ml, 6.4 mmol). The reaction was stirred to 25° C. over 16 h. Water (100 ml) and ethyl acetate (100 ml) were added and the phases were separated. The aqueous phase was extracted with ethyl acetate (2×50 ml) and the combined organic phases were washed with a saturated aqueous sodium chloride solution (25 ml). After drying over sodium sulfate and evaporation the residue was purified by silica gel chromatography (5-80% ethyl acetate/hexanes) to give 0.39 g (53%) of 2-chloro-N-(1-ethyl-1H-pyrazol-5-yl)-N-isopropylacetamide as a brownish solid. MS (ESI) m/z 230 [M+H]⁺.

### Step 3

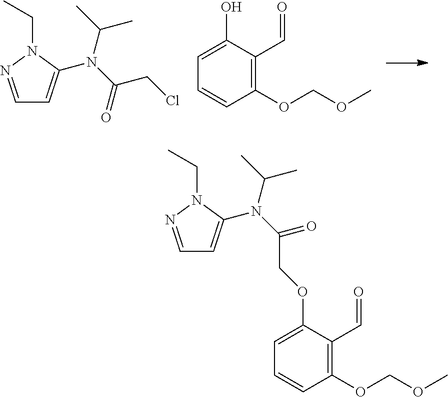

2-Chloro-N-(1-ethyl-1H-pyrazol-5-yl)-N-isopropylacetamide (0.39 g, 1.7 mmol) and 2-hydroxy-6-(methoxymethoxy)benzaldehyde (0.309 g, 1.7 mmol) were dissolved in DMF (8.5 ml) and purged with N₂ gas. Potassium carbonate (0.47 g, 3.4 mmol) was added and the mixture was stirred in a heat block at 60° C. After 2 h, the reaction was cooled and partitioned into ethyl acetate (100 ml) and water (100 ml). The phases were separated and the aqueous phase was extracted with ethyl acetate (2×50 ml). The combined organic phases were washed with water (50 ml) and a saturated aqueous sodium chloride solution (50 ml), and dried over sodium sulfate. After evaporation the residue was purified by silica gel chromatography (5-50% ethyl acetate/hexanes) to give 0.38 g (59%) of N-(1-ethyl-1H-pyrazol-5-yl)-2-(2-formyl-3-(methoxymethoxy)phenoxy)-N-isopropylacetamide as a faintly-colored viscous oil. MS (ESI) m/z 376 [M+H]⁺.

### Step 4

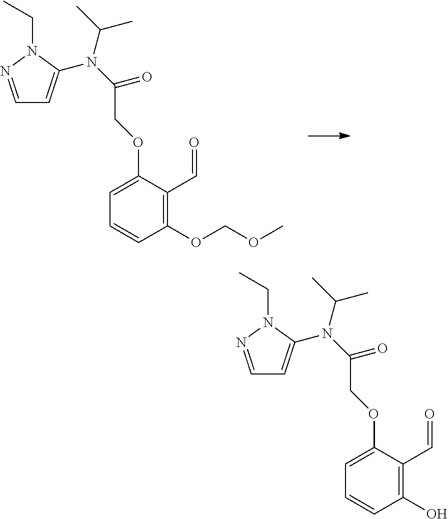

N-(1-Ethyl-1H-pyrazol-5-yl)-2-(2-formyl-3-(methoxymethoxy)phenoxy)-N-isopropylacetamide (0.38 g, 1.01 mmol) was dissolved in THF (10 ml), purged with N₂ gas and stirred in an ice bath. HCl (concentrated, 0.34 ml, 4.05 mmol) was slowly added and the solution stirred to 25° C. More HCl (0.3 ml) was added over 4 h with warming (40° C.) to reach completion of reaction. 10% Sodium bicarbonate solution (20 ml) was added and the mixture was extracted with ethyl acetate (3×75 ml). The combined organic phases were washed with a saturated aqueous sodium chloride solution (50 ml) and dried over sodium sulfate. After evaporation the resulting solid was purified by silica gel chromatography (5-80% ethyl acetate/hexanes) to give 0.179 g (53%) of N-(1-ethyl-1H-pyrazol-5-yl)-2-(2-formyl-3-hydroxyphenoxy)-N-isopropylacetamide as a white solid. ¹H NMR (400 MHz, CDCl₃) δ 11.90 (s, 1H), 10.35 (s, 1H), 7.50 (s, 1H), 7.43 (t, J=8.43 Hz, 1H), 6.67 (d, J=8.53 Hz, 1H), 6.39 (d, J=8.27 Hz, 1H), 6.32-6.27 (m, 1H), 6.12 (d, J=7.30 Hz, 1H), 5.65 (s, 1H), 4.25-4.13 (m, 3H), 1.46 (t, J=7.23 Hz, 3H), 1.22 (d, J=6.54 Hz, 3H), 1.14 (d, J=6.51 Hz, 3H). MS (ESI) m/z 332 [M+H]⁺. MP 179-182° C.

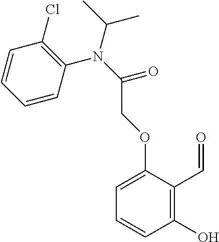 [SMILES: CC(C)N(C(=O)COc1cccc(O)c1C=O)c1ccccc1Cl]

### GBT001149 Preparation of N-(2-chlorophenyl)-2-(2-formyl-3-hydroxyphenoxy)-N-isopropylacetamide

GBT001149 was prepared using procedures similar to these described for GBT001061.

¹H NMR (400 MHz, CDCl₃) δ 11.94 (s, 1H), 10.24 (s, 1H), 7.58 (d, J=7.92 Hz, 1H), 7.45-7.30 (m, 3H), 7.24 (d, J=7.55 Hz, 1H), 6.52 (d, J=8.49 Hz, 1H), 6.21 (d, J=8.39 Hz, 1H), 4.88 (hept, J=6.64 Hz, 1H), 4.39 (q, J=15.14 Hz, 2H), 1.27 (d, J=6.68 Hz, 3H), 1.04 (d, J=6.87 Hz, 3H). MS (ESI) m/z 348 [M+H]⁺.

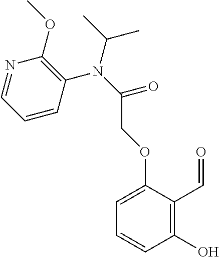 [SMILES: COc1ncccc1N(C(=O)COc1cccc(O)c1C=O)C(C)C]

### GBT001150 Preparation of 2-(2-formyl-3-hydroxyphenoxy)-N-isopropyl-N-(2-methoxypyridin-3-yl)acetamide

GBT001150 was prepared using procedures similar to these described for GBT001061.

¹H NMR (400 MHz, CDCl₃) δ 11.93 (s, 1H), 10.22 (s, 1H), 8.28-8.21 (m, 1H), 7.43-7.30 (m, 2H), 6.98 (dd, J=5.33, 7.49 Hz, 1H), 6.52 (d, J=8.46 Hz, 1H), 6.20 (d, J=8.29 Hz, 1H), 4.92 (hept, J=6.78 Hz, 1H), 4.38 (s, 2H), 4.00 (s, 3H), 1.16 (d, J=6.73 Hz, 3H), 1.00 (d, J=6.83 Hz, 3H). MS (ESI) m/z 343 [M+H]⁺.

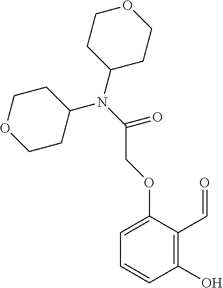 [SMILES: O=Cc1c(O)cccc1OCC(=O)N(C1CCOCC1)C1CCOCC1]

### GBT001203 Preparation of 2-(2-formyl-3-hydroxyphenoxy)-N,N-bis(tetrahydro-2H-pyran-4-yl)acetamide

GBT001203 was prepared using procedures similar to these described for GBT001061.

¹H NMR (400 MHz, CDCl₃) δ 11.91 (s, 1H), 10.39 (s, 1H), 7.40 (dd, J=8.22 Hz, 1H), 6.58 (d, J=8.85 Hz, 1H), 6.42 (d, J=8.01 Hz, 1H), 4.78 (s, 2H), 4.09-4.00 (m, 4H), 3.83 (s, 1H), 3.40-3.35 (m, 5H), 2.82 (s, 2H), 1.94 (s, 2H), 1.60 (s, 2H), 1.35 (s, 2H). MS (ESI) m/z 364 [M+H]⁺. MP 272-275° C.

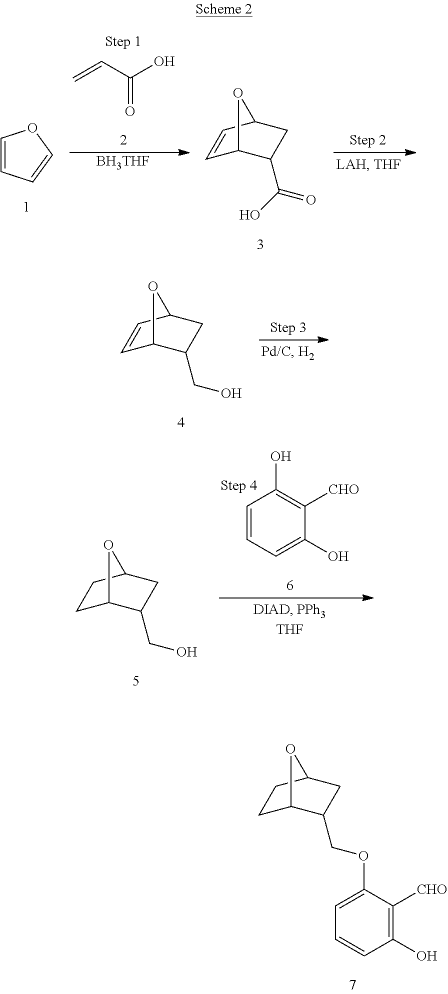

### GBT001027 Preparation of 2-(((1S,2R,4R)-7-oxabicyclo[2.2.1]heptan-2-yl)methoxy)-6-hydroxybenzaldehyde

GBT001027 was synthesized according to Scheme 2.

### Step 1

Synthesis of (1S,2S,4S)-7-oxabicyclo[2.2.1]hept-5-ene-2-carboxylic acid. Into a 25-mL round-bottom flask, was placed furan (8.16 g, 119.87 mmol, 3.00 equiv), prop-2-enoic acid (2.88 g, 39.97 mmol, 1.00 equiv). This was followed by the addition of BH₃THF (1.0M in tetrahydrofuran) (0.9 mL, 0.02 equiv) dropwise with stirring at −5° C. 0.45 mL of BH₃THF was added first. Another 0.45 mL added after 16 hours. The resulting solution was stirred for 24 h at −2° C. The solids were collected by filtration. The filter cake was washed with 60 mL hexane (0° C.). This provided 2.93 g (52%) of 7-oxabicyclo[2.2.1]hept-5-ene-2-carboxylic acid as a white solid.

### Step 2

Into a 100-mL round-bottom flask, was placed LiAlH₄ (780 mg, 20.55 mmol, 2.50 equiv) in tetrahydrofuran (30 mL). This was followed by the addition of a solution of 7-oxabicyclo[2.2.1]hept-5-ene-2-carboxylic acid (1.15 g, 8.21 mmol, 1.00 equiv) in tetrahydrofuran (10 mL) dropwise with stirring at 0° C. The resulting solution was stirred for 8 h at 0° C. The reaction was then quenched by the addition of 0.78 mL of water, 0.78 mL of 2.5M sodium hydroxide aq., and 2.3 mL of water. The resulting solution was diluted with 40 mL of ethyl acetate. The solids were filtered out. The resulting mixture was concentrated under vacuum. The residue was applied onto a silica gel column with ethyl acetate/petroleum ether (1:3-1:1) as eluent to furnish 0.93 g (90%) of 7-oxabicyclo[2.2.1]hept-5-en-2-ylmethanol as a colorless oil.

### Step 3

Into a 25-mL round-bottom flask, was placed 7-oxabicyclo[2.2.1]hept-5-en-2-ylmethanol (270 mg, 2.14 mmol, 1.00 equiv) in ethanol (10 mL). Palladium carbon (10%, 30 mg, 0.10 equiv) was added to the reaction. The resulting solution was stirred for 5 h at room temperature under 1 atm of hydrogen atmosphere. Upon completion of reaction, the solids were filtered out. The resulting mixture was concentrated under vacuum to provide 250 mg (91%) of 7-oxabicyclo[2.2.1]heptan-2-ylmethanol as a colorless oil.

### Step 4

Into a 25-mL round-bottom flask, was placed 7-oxabicyclo[2.2.1]heptan-2-ylmethanol (240 mg, 1.87 mmol, 1.00 equiv), 2,6-dihydroxybenzaldehyde (323 mg, 2.34 mmol, 1.25 equiv), PPh₃ (613 mg, 2.34 mmol, 1.25 equiv) in tetrahydrofuran (10 mL). This was followed by the addition of DIAD (473 mg, 2.34 mmol, 1.25 equiv) dropwise with stirring at 0° C. The resulting solution was stirred for 30 min at 0° C. and for an additional 1 h at room temperature. The reaction was then quenched by the addition of 10 mL of water. The resulting solution was extracted with 60 mL of ethyl acetate and the organic layers combined. The resulting mixture was washed with 3×20 mL of water and 2×20 mL of sodium chloride aq. The mixture was dried over anhydrous sodium sulfate. The residue was applied onto a silica gel column with ethyl acetate/petroleum ether (1:15) as eluent. The crude product was purified by Prep-HPLC with the following conditions (Prep-HPLC-010): Column, SunFire Prep C₁₈ OBD Column, 5 um, 19*150 mm; mobile phase, water with 0.05% TFA and MeCN (hold 45.0% MeCN in 10 min, up to 95.0% in 2 min, down to 45.0% in 2 min); Detector, Waters2545 UvDector 254&220 nm. This provided 215 mg (46%) of 2-hydroxy-6-[7-oxabicyclo[2.2.1]heptan-2-ylmethoxy]benzaldehyde as a white solid.

¹HNMR (400 MHz, CDCl₃, ppm): 11.90 (s, 1H), 10.34 (s, 1H), 7.38 (t, J=8.4 Hz, 1H), 6.54 (d, J=8.8 Hz, 1H), 6.34 (d, J=8.0 Hz, 1H), 4.70-4.56 (m, 2H), 4.22-4.13 (m, 1H), 3.92 (t, J=9.6 Hz, 1H), 2.72-2.60 (m, 1H), 2.10-2.00 (m, 1H), 1.85-1.65 (m, 3H), 1.48-1.38 (m, 1H), 1.09 (dd, J=12.0 Hz, 5.2 Hz, 1H); LSMS (ES, m/z) 249.0 [M+1]⁺

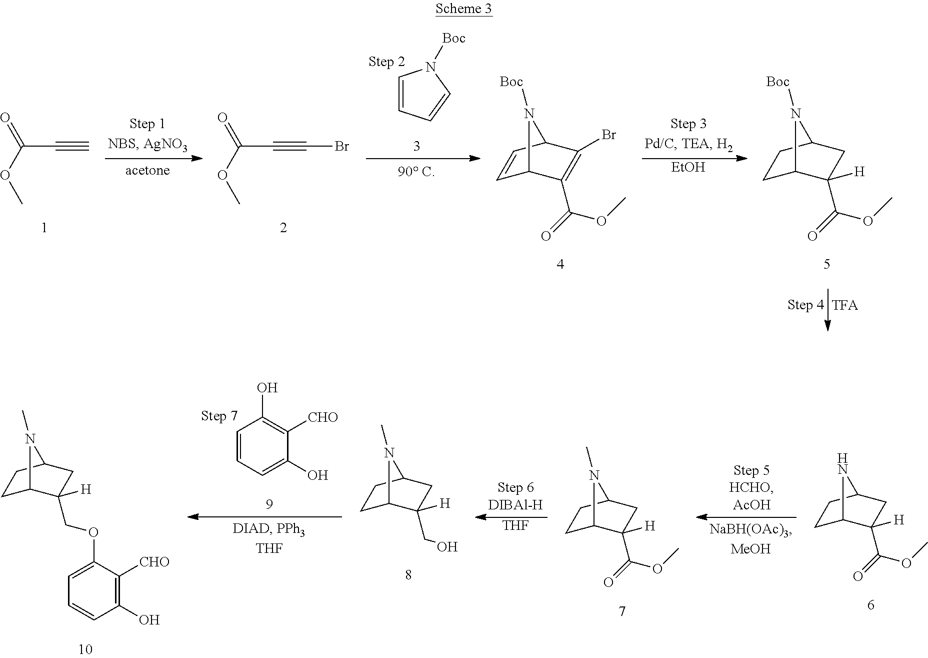

### GBT001092 Preparation of 2-hydroxy-6-(((1S,2S,4R)-7-methyl-7-azabicyclo[2.2.1]heptan-2-yl)methoxy)benzaldehyde

GBT001092 was prepared according to the seven-step synthetic sequence described in Scheme 3.

### Step 1

Into a 250-mL round-bottom flask, was placed a solution of methyl prop-2-ynoate (1) (4.9 g, 58.28 mmol, 1.00 equiv) in acetone (dried over magnesium sulfate) (120 mL). NBS (15 g, 84.28 mmol, 1.40 equiv) and AgNO₃ (1.0 g, 0.10 equiv) were added to the reaction. The resulting solution was stirred for 20 h at room temperature. The solids were filtered out. The resulting mixture was concentrated under vacuum. The crude product was purified by distillation and the fraction was collected at 40-55° C. This provided 4.7 g (49%) of methyl 3-bromoprop-2-ynoate (2) as a colorless oil.

### Step 2

Into a 50-mL round-bottom flask, was placed tert-butyl 1H-pyrrole-1-carboxylate (3) (14.4 g, 86.12 mmol, 3.00 equiv), methyl 3-bromoprop-2-ynoate (2) (4.7 g, 28.84 mmol, 1.00 equiv). The resulting solution was stirred for 20 h at 90° C. The residue was applied onto a silica gel column with ethyl acetate/petroleum ether (1:30˜1:15) as eluent to furnish 2.89 g (30%) of 7-tert-butyl 2-methyl 3-bromo-7-azabicyclo[2.2.1]hepta-2,5-diene-2,7-dicarboxylate (4) as a yellow oil.

### Step 3

Into a 100-mL round-bottom flask, was placed a solution of 7-tert-butyl 2-methyl 3-bromo-7-azabicyclo[2.2.1]hepta-2,5-diene-2,7-dicarboxylate (4) (2.3 g, 6.97 mmol, 1.00 equiv) in methanol (40 mL). Palladium carbon (10% contains H₂O) (230 mg, 0.10 equiv) and TEA (1.78 g, 17.59 mmol, 2.50 equiv) were added to the reaction mixture. The resulting solution was stirred for 5 h at room temperature under 1 atm of hydrogen atmosphere. The solids were filtered out. The resulting mixture was concentrated under vacuum. The resulting solution was diluted with 150 mL of ethyl acetate, and then it was washed with 2×50 mL of water. The mixture was dried over anhydrous sodium sulfate and concentrated under vacuum. The residue was applied onto a silica gel column with ethyl acetate/petroleum ether (1:30) as eluent to provided 1.52 g (85%) of 7-tert-butyl 2-methyl 7-azabicyclo[2.2.1]heptane-2,7-dicarboxylate (5) as a colorless oil.

### Step 4

Into a 50-mL round-bottom flask, was placed 7-tert-butyl 2-methyl 7-azabicyclo[2.2.1]heptane-2,7-dicarboxylate (5) (550 mg, 2.15 mmol, 1.00 equiv), dichloromethane (20 mL) and trifluoroacetic acid (2 mL). The resulting solution was stirred for 1 h at room temperature. The reaction was then quenched by the addition of 30 mL of sodium carbonate (sat. aq.). The resulting solution was extracted with 2×40 mL of dichloromethane and the organic layers combined. The resulting mixture was washed with 2×40 mL of brine, dried over anhydrous sodium sulfate and concentrated under vacuum. This provided 310 mg (93%) of methyl 7-azabicyclo[2.2.1]heptane-2-carboxylate (6) as a brown oil.

### Step 5

Into a 50-mL round-bottom flask, was placed methyl 7-azabicyclo[2.2.1]heptane-2-carboxylate (6) (310 mg, 2.00 mmol, 1.00 equiv), methanol (20 mL), HCHO (40% aq) (0.5 mL), acetic acid (0.1 mL). This was followed by the addition of NaBH(OAc)₃ (1.06 g, 2.50 equiv) at 0° C. The resulting solution was stirred for 40 min at 0° C., and then it was quenched by the addition of 10 mL of sodium carbonate aq. The resulting solution was extracted with 3×40 mL of dichloromethane and the organic layers combined. The resulting mixture was washed with 2×50 mL of brine, dried over anhydrous sodium sulfate and concentrated under vacuum. This provided 246 mg (73%) of methyl 7-methyl-7-azabicyclo[2.2.1]heptane-2-carboxylate (7) as a brown oil. Step 6. Into a 25-mL round-bottom flask, was placed a solution of methyl 7-methyl-7-azabicyclo[2.2.1]heptane-2-carboxylate (7) (246 mg, 1.45 mmol, 1.00 equiv) in tetrahydrofuran (10 mL). This was followed by the addition of DIBAl-H (1.0 M in hexane) (5.8 mL, 2.00 equiv) dropwise with stirring at −40° C. The resulting solution was stirred for 1 h at −40° C., and then it was quenched by the addition of 1.0 mL of 2.5M sodium hydroxide aq. The resulting solution was diluted with 10 mL of DCM. The solids were filtered out. The filter cake was washed by THF and DCM twice. The filtrate was concentrated under vacuum to yield 205 mg (100%) of [7-methyl-7-azabicyclo[2.2.1]heptan-2-yl]methanol (8) as a light yellow solid.

### Step 7

Into a 25-mL round-bottom flask, was placed [7-methyl-7-azabicyclo[2.2.1]heptan-2-yl]methanol (8) (200 mg, 1.42 mmol, 1.00 equiv), 2,6-dihydroxybenzaldehyde (9) (400 mg, 2.90 mmol, 1.50 equiv), PPh₃ (760 mg, 2.90 mmol, 1.50 equiv), tetrahydrofuran (10 mL). This was followed by the addition of DIAD (586 mg, 2.90 mmol, 1.50 equiv) dropwise with stirring at 0° C. The resulting solution was stirred for 30 min at 0° C. and for an additional 1 h at room temperature. The reaction was then quenched by the addition of 10 mL of water. The resulting solution was extracted with 3×30 mL of ethyl acetate and the organic layers combined. The resulting mixture was washed with 2×30 mL of water and 1×30 mL of sodium chloride aq. The mixture was dried over anhydrous sodium sulfate. The crude product was purified by Prep-HPLC with the following conditions (Prep-HPLC-010): Column, Gemini-NX 150*21.20 mm C₁₈ AXIA Packed, 5 um 110A; mobile phase, water with 0.05% TFA and MeCN (15.0% MeCN up to 32.0% in 6 min); Detector, 254 nm. This provided 94 mg (25%) of 2-hydroxy-6-([7-methyl-7-azabicyclo[2.2.1]heptan-2-yl]methoxy)benzaldehyde (10) as a light yellow solid.

¹HNMR (400 MHz, CDCl₃, ppm): 13.20 (br s, 1H), 11.90 (s, 1H), 10.29 (s, 1H), 7.45-7.43 (m, 1H), 6.62-6.58 (m, 1H), 6.38-6.35 (m, 1H), 4.30-3.90 (m, 4H), 3.40-3.30 (m, 1H), 2.80 (s, 3H), 2.70-2.65 (m, 1H), 2.50-1.95 (m, 3H), 1.80-1.60 (m, 1H), 1.50-1.30 (m, 1H). MS (ES, m/z) 262.1 [M-CF₃COOH+1]⁺.

What is claimed is:

1. A compound of formula (I): 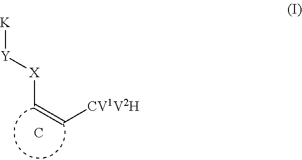
or a tautomer thereof, or a pharmaceutically acceptable salt of each thereof, wherein
K is 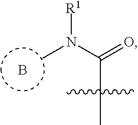
or K is: 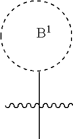
ring B is C₆-C₁₀ aryl, C₃-C₈ cycloalkyl, a 5-10 membered heteroaryl containing up to 5 ring heteroatoms or a 4-10 membered heterocycle containing up to 5 ring heteroatoms, wherein the heteroatom is selected from the group consisting of O, N, S, and oxidized forms of N and S, wherein each of the aryl, heteroaryl, cycloalkyl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
ring B¹ is a 5-10 membered heteroaryl containing up to 5 ring heteroatoms or 4-10 membered heterocycle containing up to 5 ring heteroatoms, wherein the heteroatom is selected from the group consisting of O, N, S, and oxidized forms of N and S, wherein at least one of the heteroatoms or oxidized forms thereof is gamma (γ) to the position where Y is attached to B, each of the heteroaryl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
each X and Y is independently CR¹⁰R¹¹, O, S, SO, SO₂, or NR¹⁰; each R¹⁰ and R¹¹ independently is hydrogen or C₁-C₃ alkyl optionally substituted with 1-3 halo, OH, or C₁-C₆ alkoxy, or CR¹⁰R¹¹ is C═O, provided that if one of Y and Z is O, S, SO, SO₂, then the other is not CO, and Y and Z are both not heteroatoms or oxidized forms thereof;
ring C is C₆-C₁₀ aryl or a 5-10 membered heteroaryl containing up to 5 ring heteroatoms, wherein the heteroatom is selected from the group consisting of O, N, S, and oxidized forms of N and S, each of which is optionally substituted with 1-4: halo, oxo, —OR², C₁-C₆ alkyl, and/or C₁-C₆ alkoxy, wherein the C₁-C₆ alkyl is optionally substituted with 1-5 halo, C₁-C₆ alkoxy and/or a 4-10 membered heterocycle containing up to 5 ring heteroatoms, wherein the heteroatom is selected from the group consisting of O, N, S, and oxidized forms of N and S;
R¹ is optionally substituted C₁-C₆ alkyl, C₂-C₆ alkenyl, or C₂-C₆ alkynyl, or is C₆-C₁₀ aryl, a 5-10 membered heteroaryl, containing up to 5 ring heteroatoms wherein the heteroatom is selected from the group consisting of O, N, S and oxidized forms of N and S, C₃-C₈ cycloalkyl or a 4-10 membered heterocycle containing up to 5 ring heteroatoms, wherein the heteroatom is selected from the group consisting of O, N, S, and oxidized forms of N and S; and
R² is hydrogen or a prodrug moiety R;
V¹ and V² independently are C₁-C₆ alkoxy; or V¹ and V² together with the carbon atom they are attached to form a ring of formula: 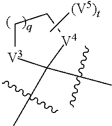
wherein each V³ and V⁴ are independently O, S, or NH, provided that when one of V³ and V⁴ is S, the other is NH, and provided that V³ and V⁴ are both not NH; q is 1 or 2; each V⁵ is independently C₁-C₆ alkyl optionally substituted with 1-3 OH groups, or V⁵ is CO₂R⁶⁰, where each R⁶⁰ independently is C₁-C₆ alkyl or hydrogen; t is 0, 1, 2, or 4; or CV¹V² is C═V, wherein V is O, NOR⁸⁰, or NNR⁸¹R⁸²;
R⁸⁰ is optionally substituted C₁-C₆ alkyl;
R⁸¹ and R⁸² independently are selected from the group consisting of hydrogen, optionally substituted C₁-C₆ alkyl, COR⁸³, or CO₂R⁸⁴;
R⁸³ is hydrogen or optionally substituted C₁-C₆ alkyl; and
R⁸⁴ is optionally substituted C₁-C₆ alkyl.

2. A compound of formula (II) of claim 1: 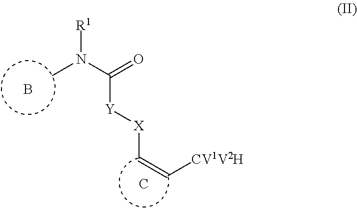
or a tautomer thereof, or a pharmaceutically acceptable salt of each thereof, wherein
ring B is C₆-C₁₀ aryl, C₃-C₈ cycloalkyl, a 5-10 membered heteroaryl containing up to 5 ring heteroatoms or a 4-10 membered heterocycle containing up to 5 ring heteroatoms, wherein the heteroatom is selected from the group consisting of O, N, S, and oxidized forms of N and S, wherein each of the aryl, heteroaryl, cycloalkyl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
each X and Y is independently CR¹⁰R¹¹, O, S, SO, SO₂, or NR¹⁰; each R¹⁰ and R¹¹ independently is hydrogen or C₁-C₃ alkyl optionally substituted with 1-3 halo, OH, or C₁-C₆ alkoxy, or CR¹⁰R¹¹ is C═O, provided that if one of Y and Z is O, S, SO, SO₂, then the other is not CO, and Y and Z are both not heteroatoms or oxidized forms thereof;
ring C is C₆-C₁₀ aryl or a 5-10 membered heteroaryl containing up to 5 ring heteroatoms, wherein the heteroatom is selected from the group consisting of O, N, S, and oxidized forms of N and S, each of which is optionally substituted with 1-4: halo, oxo, —OR², C₁-C₆ alkyl, and/or C₁-C₆ alkoxy, wherein the C₁-C₆ alkyl is optionally substituted with 1-5 halo, C₁-C₆ alkoxy and/or a 4-10 membered heterocycle containing up to 5 ring heteroatoms, wherein the heteroatom is selected from the group consisting of O, N, S, and oxidized forms of N and S;
R¹ is optionally substituted C₁-C₆ alkyl, C₂-C₆ alkenyl, or C₂-C₆ alkynyl, or is C₆-C₁₀ aryl, a 5-10 membered heteroaryl, containing up to 5 ring heteroatoms wherein the heteroatom is selected from the group consisting of O, N, S and oxidized forms of N and S, C₃-C₈ cycloalkyl or a 4-10 membered heterocycle containing up to 5 ring heteroatoms, wherein the heteroatom is selected from the group consisting of O, N, S, and oxidized forms of N and S; and
R² is hydrogen or a prodrug moiety R;
V¹ and V² independently are C₁-C₆ alkoxy; or V¹ and V² together with the carbon atom they are attached to form a ring of formula: 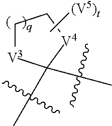
wherein each V³ and V⁴ are independently 0, S, or NH, provided that when one of V³ and V⁴ is S, the other is NH, and provided that V³ and V⁴ are both not NH; q is 1 or 2; each V⁵ is independently C₁-C₆ alkyl optionally substituted with 1-3 OH groups, or V⁵ is CO₂R⁶⁰, where each R⁶⁰ independently is C₁-C₆ alkyl or hydrogen; t is 0, 1, 2, or 4; or CV¹V² is C═V, wherein V is O, NOR⁸⁰, or NNR⁸¹R⁸²;
R⁸⁰ is optionally substituted C₁-C₆ alkyl;
R⁸¹ and R⁸² independently are selected from the group consisting of hydrogen, optionally substituted C₁-C₆ alkyl, COR⁸³, or CO₂R⁸⁴;
R⁸³ is hydrogen or optionally substituted C₁-C₆ alkyl; and
R⁸⁴ is optionally substituted C₁-C₆ alkyl.

3. The compound of claim 2, wherein CV¹V² is C═V, wherein V is O, and wherein the remaining variables are defined as in claim 2.

4. The compound of claim 3, of formula (III): 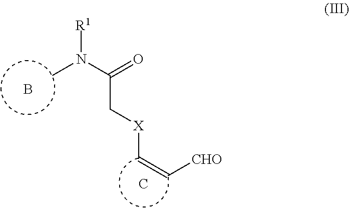
wherein the remaining variables are defined as in claim 3.

5. A compound of claim 3 of formula (IV): 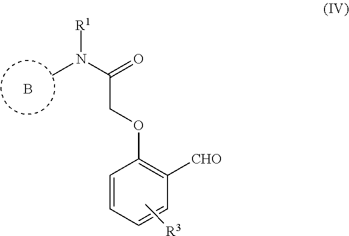
wherein
R¹ is optionally substituted C₁-C₆ alkyl or a 4-10 membered heterocycle containing up to 5 ring heteroatoms, wherein the heteroatom is selected from the group consisting of O, N, S;
R³ is halo, oxo, C₁-C₆ alkyl and/or C₁-C₆ alkoxy; and
R⁴ is hydrogen or a prodrug moiety R.

6. The compound of claim 5, wherein ring B is
C₃-C₈ heteroaryl containing 1-3 heteroatoms, wherein the heteroaryl is optionally substituted with C₁-C₆ alkyl or C₁-C₆ alkoxy;
phenyl substituted with 1-3 halo, or
C₃-C₈ heterocyclyl containing 1-3 heteroatoms.

7. A compound of claim 1 selected from the group consisting of: 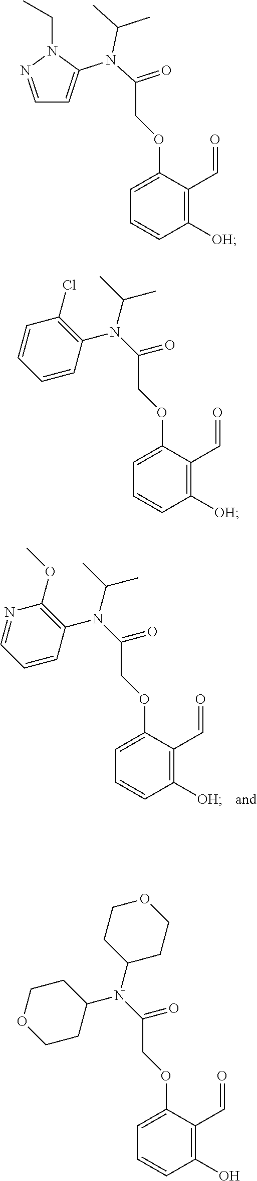 [SMILES: CCn1nccc1N(C(=O)COc1cccc(O)c1C=O)C(C)C ; CC(C)N(C(=O)COc1cccc(O)c1C=O)c1ccccc1Cl ; COc1ncccc1N(C(=O)COc1cccc(O)c1C=O)C(C)C ; O=Cc1c(O)cccc1OCC(=O)N(C1CCOCC1)C1CCOCC1]
or N oxides thereof, or a pharmaceutically acceptable salt of each thereof.

8. A compound of formula (V) of claim 1: 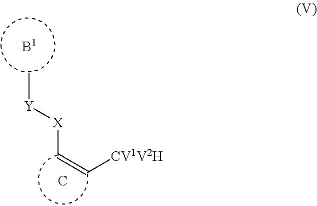
or a tautomer thereof, or a pharmaceutically acceptable salt of each thereof, wherein
ring B¹ is a 5-10 membered heteroaryl containing up to 5 ring heteroatoms or 4-10 membered heterocycle containing up to 5 ring heteroatoms, wherein the heteroatom is selected from the group consisting of O, N, S, and oxidized forms of N and S, wherein at least one of the heteroatoms or oxidized forms thereof is γ to the position where Y is attached to B¹, each of the heteroaryl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
each X and Y is independently CR¹⁰R¹¹, O, S, SO, SO₂, or NR¹⁰; each R¹⁰ and R¹¹ independently is hydrogen or C₁-C₃ alkyl optionally substituted with 1-3 halo, OH, or C₁-C₆ alkoxy, or CR¹⁰R¹¹ is C═O, provided that if one of Y and Z is O, S, SO, SO₂, then the other is not CO, and Y and Z are both not heteroatoms or oxidized forms thereof;
ring C is C₆-C₁₀ aryl or a 5-10 membered heteroaryl containing up to 5 ring heteroatoms, wherein the heteroatom is selected from the group consisting of O, N, S, and oxidized forms of N and S, each of which is optionally substituted with 1-4: halo, oxo, —OR², C₁-C₆ alkyl, and/or C₁-C₆ alkoxy, wherein the C₁-C₆ alkyl is optionally substituted with 1-5 halo, C₁-C₆ alkoxy and/or a 4-10 membered heterocycle containing up to 5 ring heteroatoms, wherein the heteroatom is selected from the group consisting of O, N, S, and oxidized forms of N and S;
R² is hydrogen or a prodrug moiety R;
V¹ and V² independently are C₁-C₆ alkoxy; or V¹ and V² together with the carbon atom they are attached to form a ring of formula: 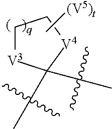
wherein each V³ and V⁴ are independently O, S, or NH, provided that when one of V³ and V⁴ is S, the other is NH, and provided that V³ and V⁴ are both not NH; q is 1 or 2; each V⁵ is independently C₁-C₆ alkyl optionally substituted with 1-3 OH groups or V⁵ is CO₂R⁶⁰, where each R⁶⁰ independently is C₁-C₆ alkyl or hydrogen; t is 0, 1, 2, or 4; or CV¹V² is C═V, wherein V is O, NOR⁸⁰, or NNR⁸¹R⁸²;
R¹¹ is optionally substituted C₁-C₆ alkyl;
R⁸¹ and R⁸² independently are selected from the group consisting of hydrogen, optionally substituted C₁-C₆ alkyl, COR⁸³, or CO₂R⁸⁴;
R⁸³ is hydrogen or optionally substituted C₁-C₆ alkyl; and
R⁸⁴ is optionally substituted C₁-C₆ alkyl.

9. The compound of claim 8, wherein CV¹V² is C═V, wherein V is O, and wherein the remaining variables are defined as in claim 8.

10. The compound of claim 9, of formula: 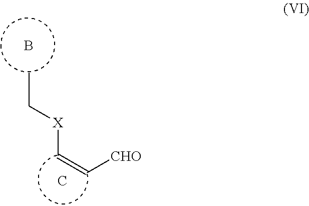
or a tautomer thereof, or a pharmaceutically acceptable salt of each thereof, wherein
ring B is a 5-10 membered heteroaryl containing up to 5 ring heteroatoms or 4-10 membered heterocycle containing up to 5 ring heteroatoms, wherein the heteroatom is selected from the group consisting of O, N, S, and oxidized forms of N and S, wherein each of the heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
X is O, S, SO or SO₂;
ring C is C₆-C₁₀ aryl or a 5-10 membered heteroaryl containing up to 5 ring heteroatoms, wherein the heteroatom is selected from the group consisting of O, N, S, and oxidized forms of N and S, each of which is optionally substituted with 1-4: halo, oxo, —OR², C₁-C₆ alkyl, and/or C₁-C₆ alkoxy, wherein the C₁-C₆ alkyl is optionally substituted with 1-5 halo, C₁-C₆ alkoxy and/or a 4-10 membered heterocycle containing up to 5 ring heteroatoms, wherein the heteroatom is selected from the group consisting of O, N, S, and oxidized forms of N and S; and
R² is hydrogen or a prodrug moiety R.

11. The compound of claim 9 of formula: 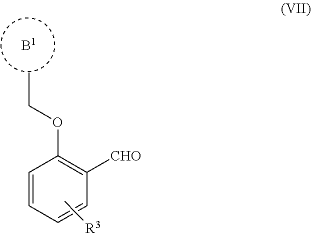
or a tautomer thereof, or a pharmaceutically acceptable salt of each thereof, wherein
ring B¹ is a 5-10 membered heteroaryl containing up to 5 ring heteroatoms or 4-10 membered heterocycle containing up to 5 ring heteroatoms, wherein the heteroatom is selected from the group consisting of O, N, S, and oxidized forms of N and S, wherein each of the aryl, heteroaryl, cycloalkyl or heterocycle is optionally substituted with 1-4: halo, C₁-C₆ alkyl, or C₁-C₆ alkoxy, wherein the C₁-C₆ alkyl is optionally substituted with 1-5 halo, C₁-C₆ alkoxy, and/or C₃-C₁₀ cycloalkyl;
R³ is halo, oxo, C₁-C₆ alkyl and/or C₁-C₆ alkoxy; and
R⁴ is hydrogen or a prodrug moiety R.

12. The compound of claim 11, wherein ring B¹ is selected from the group consisting of 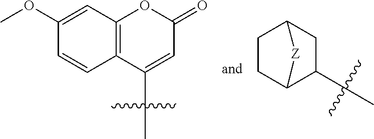
wherein Z is O or NR¹⁰; and
R¹⁰ is hydrogen or optionally substituted C₁-C₆ alkyl.

13. A compound of claim 1 selected from the group consisting of: 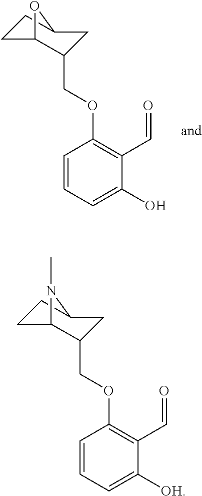 [SMILES: O=Cc1c(O)cccc1OCC1CC2CCC1O2 ; CN1C2CCC1C(COc1cccc(O)c1C=O)C2]
or N oxides thereof, or a pharmaceutically acceptable salt of each thereof.

14. A compound of formula (VIII): 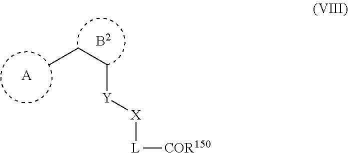
or a tautomer thereof, or a pharmaceutically acceptable salt of each thereof, wherein
ring A is a 5-10 membered heteroaryl, wherein the heteroatom is selected from the group consisting of O, N, S, and oxidized forms of N and S, wherein the heteroaryl is optionally substituted with 1-4: C₁-C₆ alkyl, wherein the C₁-C₆ alkyl is optionally substituted with 1-5 halo, C₁-C₆ alkoxy, and/or C₃-C₁₀ cycloalkyl;
ring B² is a 5-10 membered heteroaryl containing up to 5 ring heteroatoms, wherein the heteroatom is selected from the group consisting of O, N, S, and oxidized forms of N and S, wherein the heteroaryl is optionally substituted with with 1-4: C₁-C₆ alkyl, wherein the C₁-C₆ alkyl is optionally substituted with 1-5 halo, C₁-C₆ alkoxy, and/or C₃-C₁₀ cycloalkyl;
each X and Y is independently CR¹⁰R¹¹, O, S, SO, SO₂, or NR¹⁰; each R¹⁰ and R¹¹ independently is hydrogen or C₁-C₃ alkyl optionally substituted with 1-3 halo, OH, or C₁-C₆ alkoxy, or CR¹⁰R¹¹ is C═O, provided that if one of Y and Z is O, S, SO, SO₂, then the other is not CO, and Y and Z are both not heteroatoms or oxidized forms thereof;
wherein Y is α or β substituted relative to ring B;
L is joined with X and is a bond or is C₁-C₆ alkylene; and
R¹⁵⁰ is hydrogen, optionally substituted C₁-C₆ alkyl, C₂-C₆ alkynyl, or C₂-C₆ alkynyl, or is C₆-C₁₀ aryl, a 5-10 membered heteroaryl, containing up to 5 ring heteroatoms wherein the heteroatom is selected from the group consisting of O, N, S, C₃-C₈ cycloalkyl or a 4-10 membered heterocycle containing up to 5 ring heteroatoms, wherein the heteroatom is selected from the group consisting of O, N, S.

15. The compound of claim 14 of formula: 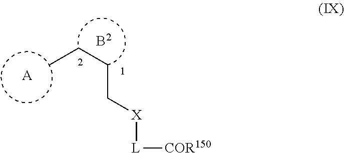
or a tautomer thereof, or a pharmaceutically acceptable salt of each thereof, wherein
ring A is a 5-10 membered heteroaryl, wherein the heteroatom is selected from the group consisting of O, N, S, and oxidized forms of N and S, wherein the heteroaryl is optionally substituted with 1-4: C₁-C₆ alkyl, wherein the C₁-C₆ alkyl is optionally substituted with 1-5 halo, C₁-C₆ alkoxy, and/or C₃-C₁₀ cycloalkyl;
ring B² is a 5-10 membered heteroaryl containing up to 5 ring heteroatoms, wherein the heteroatom is selected from the group consisting of O, N, S, and oxidized forms of N and S, wherein the heteroaryl is optionally substituted with with 1-4: C₁-C₆ alkyl, wherein the C₁-C₆ alkyl is optionally substituted with 1-5 halo, C₁-C₆ alkoxy, and/or C₃-C₁₀ cycloalkyl;
X is O, S, SO or SO₂;
L is joined with X and is a bond or is C₁-C₆ alkylene; and
R¹⁵⁰ is hydrogen or optionally substituted C₁-C₆ alkyl.

16. The compound of claim 15, wherein L is methylene or ethylene.

17. A compound of claim 14 selected from:

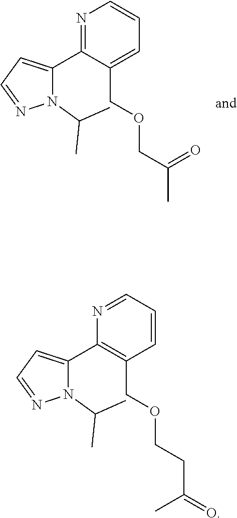 [SMILES: CC(=O)COCc1cccnc1-c1ccnn1C(C)C ; CC(=O)CCOCc1cccnc1-c1ccnn1C(C)C]

18. A composition comprising a compound of claim 2 and at least one pharmaceutically acceptable excipient.

19. A method for increasing oxygen affinity of hemoglobin S in a subject, the method comprising administering to a subject in need thereof a therapeutically effective amount of a compound of claim 2.

20. A method for treating oxygen deficiency associated with sickle cell anemia, the method comprising administering to a subject in need thereof a therapeutically effective amount of a compound of claim 2.
